SEQUENCE LISTING (15 sequences)

SEQ ID NO: 1 - protein, 56 aa, Nicotiana benthamiana
AARRPPPPPPTSEEKKDPNMSGVMAKVLASKRRKEAMKESIAKLREKGKPVKEPSQ

SEQ ID NO: 2 - protein, 112 aa, Nicotiana benthamiana
MARSLSPIAAATTLASKSIPLAFHDKKMDTTLLSRRSLALGLAGVVLNAGNNNANAAARRPPPPPPTSEEKKDPNMSGVMAKVLASKRRKEAMKESIAKLREKGKPVKEPSQ

SEQ ID NO: 3 - protein, 55 aa, Solanum tuberosum
AARRPPPPPPTEKKDPNVSGVLAKVLASKRRKEAMKESIAKLREKGKPVKEVPSE

SEQ ID NO: 4 - protein, 120 aa, Solanum tuberosum
MAQSVSPTAAATLTSLSTKKNARLSSFKVLACQLDAKINVSRRSLALSLAGVAAALNGGNNNANAAARRPPPPPPTEKKDPNVSGVLAKVLASKRRKEAMKESIAKLREKGKPVKEVPSE

SEQ ID NO: 5 - protein, 14 aa, Nicotiana benthamiana
AKVLASKRRKEAMK

SEQ ID NO: 6 - DNA, 889 bp, Nicotiana benthamiana
atggctcggtcattgtctccaattgcagcagctacgacattggcctcgaagagtatccctttggcttttcatgataaaaaaatggatacaactcttctctctcgcaggagtcttgccttgggcttggccggagttgtgctaaatgcaggtaacaacaatgcaaatgctgcagcaagaaggcctccaccacccccacccacatcagaggagaaaaaggacccaaatatgagtggtgtcatggctaaagtattagctagcaagagaagaaaggaagctatgaaagaatccatagccaagctaagggagaaagggaagccagtcaaagaaccatctcaatagtaatttgtaaccaatattatgttttaagacaattgatgtgttgcatgcttgctcttctttacattcttgaaattattggaagtcaaatgcaatcaataatgagatcaaaaaataagaagcgtactgcattcactttgtttatactaatgaaaatgaaaaagaagatttttgaatattttgtcgggagaagaagggatcttgtccctggtacgagttgaataatgaaggattttgcaattattctatgctaagcatgttgggaagaaaaattgaccatttcccgtaaaccccgagatcttgccccttttgtaaagagaaaaattgagaaataaaaacaacacaagaatttaacgtggaaacccaaaaccggaaaaaaaccacaaccgttgtcaaaagacaacaaccgaccactatgtgaaaaattcttacaatcacataaacctcagtacattctcctaacaaatagtgttagaaatgcattcggtttggtacatttgaaaacataacttaatcccccttatataggcaagcctccaagctactcttccac

SEQ ID NO: 7 - DNA, 339 bp, Nicotiana benthamiana
atggctcggtcattgtctccaattgcagcagctacgacattggcctcgaagagtatccctttggcttttcatgataaaaaaatggatacaactcttctctctcgcaggagtcttgccttgggcttggccggagttgtgctaaatgcaggtaacaacaatgcaaatgctgcagcaagaaggcctccaccacccccacccacatcagaggagaaaaaggacccaaatatgagtggtgtcatggctaaagtattagctagcaagagaagaaaggaagctatgaaagaatccatagccaagctaagggagaaagggaagccagtcaaagaaccatctcaatag

SEQ ID NO: 8 - DNA, 49 bp, Nicotiana benthamiana
ggggacaagtttgtacaaaaaagcaggctatggctcggtcattgtctcc

SEQ ID NO: 9 - DNA, 54 bp, Nicotiana benthamiana
gccagtcaaagaaccatctcaatagacccagctttcttgtacaaagtggtcccc

SEQ ID NO: 10 - DNA, 38 bp, Artificial Sequence
  forward primer for the Tobacco Rattle Virus (TRV) RNA2 (pJK037) construct
ggctacggtctccattcttgagatggttctttgactgg

SEQ ID NO: 11 - DNA, 35 bp, Artificial Sequence
  reverse primer for the Tobacco Rattle Virus (TRV) RNA2 (pJK037) construct
tggagacaatgaccgagcggatcgagaccgtagcc

SEQ ID NO: 12 - protein, 22 aa, Artificial Sequence
  Synthetic flg22 peptide
QRLSTGSRINSAKDDAAGLQIA

SEQ ID NO: 13 - protein, 38 aa, Solanum tuberosum
EKKDPNVSGVLAKVLASKRRKEAMKESIAKLREKGKPV

SEQ ID NO: 14 - protein, 38 aa, Nicotiana benthamiana
EKKDPNMSGVMAKVLASKRRKEAMKESIAKLREKGKPV

SEQ ID NO: 15 - protein, 126 aa, Arabidopsis thaliana
MVAHSLVPLSPAAHAARLSSPSPRSLPQAPPVVLAVPPINRRTILVGLGGALWSWNALAAKEEAMAAARRPPPPPPKEKKDPTVTGVQAKVLASKKRKEEMKASIAKLREKGKPVVEAKPSSSSSE

(19) United States
(12) Patent Application Publication
(10) Pub. No.: US 2024/0110198 A1
(43) Pub. Date: Apr. 4, 2024

(54) METHOD OF PROVIDING BROAD-SPECTRUM RESISTANCE TO PLANTS, AND PLANTS THUS OBTAINED

(71) Applicants: Erik ANDREASSON, Eslöv (SE); Marit LENMAN, Lund (SE); Muhammad Awais ZAHID, Malmö (SE); Nam PHUONG KIEU, Lund (SE); Naga Charan KONAKALLA, Alnarp (SE)

(72) Inventors: Erik ANDREASSON, Eslöv (SE); Marit LENMAN, Lund (SE); Muhammad Awais ZAHID, Malmö (SE); Nam PHUONG KIEU, Lund (SE); Naga Charan KONAKALLA, Alnarp (SE); Svante RESJÖ, Lund (SE); Ramesh VETUKURI, Malmö (SE)

(21) Appl. No.: 18/263,146

(22) PCT Filed: Dec. 30, 2021
(86) PCT No.: PCT/SE2021/051320

§ 371 Date: Jul. 27, 2023

(30) Foreign Application Priority Data
Feb. 19, 2021 (SE) 2150182-0

(51) Int. Cl. C12N 15/82; C12N 9/22

(52) U.S. Cl. CPC C12N 15/8279; C12N 9/22; C12N 15/8273; C12N 2310/20

(57) ABSTRACT

A method of obtaining a plant cell having increased pathogen resistance and/or abiotic stress tolerance is provided, the method comprising the steps of a) providing a plant cell, and b) modifying the genome of the plant cell so as to obtain a decreased or inactivated expression of a protein having an amino acid sequence in which at least one part of the amino acid sequence has at least 78%, such as at least 85%, more preferably at least 90% sequence identity to SEQ ID NO: 5, and wherein the protein comprises less than 200 amino acids. A method of obtaining a plant, as well as a plant cell and a plant, are also provided.

### TECHNICAL FIELD

The technology proposed herein relates generally to the field of plant pathogens and methods of providing broad-spectrum resistance, including pathogen resistance and/or abiotic stress tolerance, to plants, as well as plants thus obtained. More particularly, the technology proposed herein relates to methods providing a decreased or inactivated expression of a protein related to stress generation of reactive oxygen species in plants, thereby modifying plant pathogen resistance and abiotic stress tolerance.

### BACKGROUND

Plants are beset by a wide number of different pathogens including various type of microorganisms. One such pathogen is the oomycete Phytophthora infestans, a microorganism that is favored by moist and cool environments and which causes the disease late blight in for example potato and tomato plants. Late blight disease has serious economical consequences and was further a major factor in the Irish potato famines in the year 1845. Symptoms of P. infestans infection include the appearance of dark blotches (lesions) on leaves and plant stems. Later, white mold may appear on the leaves and the whole plant may quickly collapse. Infected tubers develop grey or dark patches that are reddish brown beneath the skin, and quickly decay to a foul-smelling mush caused by the infestation of secondary soft bacterial rots.

Late blight disease is difficult to control despite the use of modern fungicides. Accordingly, in many cases the infected plants and tubers need to be destroyed in the field. If infected tubers are harvested and stored together with uninfected tubers, there is a very high risk that the disease will spread leading to the loss of a major part or all of the stored tubers.

Due to the significant threat of the disease, and the estimated caused total annual losses of about § 6 billion, efforts have also been made to breed or genetically engineer plants with improved resistance to the disease. However, these efforts have so far met with limited success due to the difficulties in crossing desired potato varieties with wild type relatives which may contain the desired potential resistance genes. Further, such resistance genes may often only work against a subset of Phytophthora infestans isolates. In addition to Phytophthora infestans, there are numerous additional pathogens and diseases which continue to be a threat to the growing and farming of these and other plants. Additionally, abiotic stress such as drought and salinity affect the growth of plants.

Accordingly, there is a need for further methods of providing increased pathogen resistance and abiotic stress tolerance, i.e. broad-spectrum resistance, to plants. There is further a need for methods capable of providing resistance to a wide variety of pathogens, as well as to a wide variety of abiotic stresses.

### OBJECTS OF THE TECHNOLOGY PROPOSED HEREIN

It is accordingly a first object of the technology proposed herein to provide a method of providing pathogen resistance and/or abiotic stress tolerance in plants.

It is a further object of the technology proposed herein to provide a method of obtaining a plant having increased resistance to Phytophthora infestans.

It is yet a further object of the technology proposed herein to provide a plant cell having increased pathogen resistance and/or abiotic stress tolerance.

It is yet a further object of the technology proposed herein to provide a plant having increased pathogen resistance, especially towards the pathogen Phytophthora infestans.

### SUMMARY

At least one of the abovementioned objects, or at least one of the further objects which will become evident from the below description, is according to a first aspect of the technology proposed herein obtained by a method of obtaining a plant cell having increased pathogen resistance and/or abiotic stress tolerance, the method comprising the steps of
a) providing a plant cell, and
b) modifying the genome of said plant cell so as to obtain a decreased or inactivated expression of a protein having an amino acid sequence in which at least one part of the amino acid sequence has at least 78%, such as at least 85%, more preferably at least 90% sequence identity to SEQ ID NO: 5, and wherein the protein comprises less than 200 amino acids.

A corresponding second aspect of the technology proposed herein relates to a plant cell having increased pathogen resistance and/or abiotic stress tolerance, wherein the genome of the plant cell has been modified to obtain a decreased or inactivated expression of a protein having an amino acid sequence in which at least one part of the amino acid sequence having has at least 78%, such as at least 85%, more preferably at least 90% sequence identity to SEQ ID NO: 5, and wherein the protein comprises less than 200 amino acids. The plant cell is not exclusively obtained by means of an essentially biological process.

Accordingly, the present invention is based on the discovery by the present inventors that plant cells and plants having increased pathogen resistance and/or abiotic stress tolerance can be obtained by decreasing or inactivating the expression of a specific protein, herein called the 72 protein or Parakletos, in the genome of the plant. Specifically, as discussed in Example 1, the present inventors found that overexpression of the 72 protein decreased the pathogen resistance towards the example pathogen Phytophthora infestans, whereas a decreased or inactivated expression of the 72 protein provided increased resistance to this pathogen.

The mature 72 protein in Nicotiana benthamiana has the following amino acid sequence:

(SEQ ID NO: 1)
AARRPPPPPPTSEEKKDPNMSGVMAKVLASKRRKEAMKESIAKLREKGK
PVKEPSQ

The corresponding full sequence (including the signal peptide) of the 72 protein in Nicotiana benthamiana has the following sequence:

(SEQ ID NO: 2)
MARSLSPIAAATTLASKSIPLAFHDKKMDTTLLSRRSLALGLAGVVLNA
GNNNANAAARRPPPPPPTSEEKKDPNMSGVMAKVLASKRRKEAMKESIA
KLREKGKPVKEPSQ

Further, the present inventors noted, as discussed in Example 2, that the increased resistance in the obtained plants was obtained via a non-pathogen specific pathway, e.g., via increased concentrations of reactive oxygen species (ROS) in the modified plant cells and plants. Accordingly, the decreased or and inactivated expression of the 72 protein provided increased concentrations of ROS, which increased concentrations indicate an increased pathogen resistance to many different pathogens.

Further, as noted in Example 3 and table 1, a wide variety of higher plants, including cereals, include genes coding for plant specific variants of the 72 protein. A corresponding Solanum tuberosum specific variant (uniprot ID M1CUF4) was identified in Potato. The mature 72 protein in Solanum tuberosum has the following amino acid sequence:

(SEQ ID NO: 3)
AARRPPPPPPTEKKDPNVSGVLAKVLASKRRKEAMKESIAKLREKGKPV
KEVPSE

The corresponding full sequence (including signal peptide) has the following amino acid sequence:

(SEQ ID NO: 4)
MAQSVSPTAAATLTSLSTKKNARLSSFKVLACQLDAKINVSRRSLALSL
AGVAAALNGGNNNANAAARRPPPPPPTEKKDPNVSGVLAKVLASKRRKE
AMKESIAKLREKGKPVKEVPSE

Further studies as detailed in Example 3 and shown in table 2 revealed that plant specific variants of the 72 protein included sequences having at least 78% sequence identity to the common motif:

(SEQ ID NO: 5)
AKVLASKRRKEAMK,

which motif is found in both the 72 protein in Nicotiana benthamiana and in Solanum tuberosum. Thus, as detailed in table 2, there are more than 200 putative plant specific variants of the 72 proteins in other plants including in all cereals.

Together, these findings support the conclusion that decreased or inactivated expression of the 72 protein or a homologous protein provides increased pathogen resistance towards pathogens affected by ROS, such as Phytophthora infestans.

The generality of this mechanism is highlighted by Examples 4-7 and 9 which demonstrate that inactivation of the 72 protein is effective in providing pathogen resistance in different plants, and for different pathogens.

Additionally, as noted in Example 8, inactivation of the 72 protein further provides an increased abiotic stress tolerance in that plants in which expression of the 72 protein was decreased or inactivated had a higher tolerance to salt. Due to the generality of the mechanism, i.e. the increased ROS production, obtained by inactivating the 72 protein, this will also provide tolerance to other types of abiotic stress such as drought. Specifically, the 72 protein only has a functional role when a plant is stressed, e.g. as shown in the pathogen-, ROS- and salt-assays in the examples. Drought is also a form of stress which shows a high degree of similarity with respect to physiological, biochemical, molecular and genetic effects as compared to salt stress, and accordingly inactivation of the 72 protein will therefore provide tolerance to other types of abiotic stress such as drought.

The method of obtaining a plant cell having increased pathogen resistance and/or abiotic stress tolerance may be comprised by a method of obtaining a plant cell having broad-spectrum resistance.

It is to be understood that the term “plant cell” also encompasses the term “plant”.

As used herein, the term “plant” includes plant cells, plant protoplasts, plant cells of tissue culture from which plants can be regenerated, plant calli, plant clumps and plant cells that are intact in plants or parts of plants such as pollen, flowers, seeds, leaves, stems, and the like.

Accordingly, the method according to the first aspect of the technology proposed herein can be performed either on a plant cell, or on a plant. Further, the method can be formed on a single plant cell or plant, or on a plurality of plant cells or plants.

Pathogen resistance is encompassed by biotic stress tolerance.

The pathogen resistance may comprise resistance to microorganisms such as virus, bacteria and/or fungi, but also to aphids. Preferably, the pathogen resistance comprises resistance to oomycetes and fungi and bacteria, preferably oomycetes and fungi, most preferably oomycotes such as Phytophthora infestans.

Further, the pathogen resistance preferably comprises resistance against pathogens of the phylum Oomycota, such as Albugo, Aphanomyces, Basidiophora, Bremia, Hyaloperonospora, Pachymetra, Paraperonospora, Perofascia, Peronophythora, Peronospora, Peronosclerospora, Phytium, Phytophthora, Plasmopara, Protobremia, Pseudoperonospora, Sclerospora, Viennotia species, as well as to pathogens belonging to the Fungi.

Bacteria may comprise genera such as Erwinia, Pectobacterium, Pantoea, Agrobacterium, Pseudomonas, Ralstonia, Burkholderia, Acidovorax, Xanthomonas, Clavibacter, Streptomyces, Xylella, and Spiroplasma. Preferably, bacteria are selected from the group consisting of Xanthomonas campestris, Pseudomonas syringae, Erwinia carotovora, and Pseudomonas santomos.

Increased pathogen resistance encompasses a lower risk or occurrence of being infected by the pathogen, and/or a lower or lesser risk or occurrence of disease and/or disease symptom if infected by the pathogen. An increased pathogen resistance can be determined and quantified by comparing the risk or occurrence of infection and/or risk or occurrence of disease and/or disease symptom for the plant cell according to the first aspect of the technology proposed herein, with those of a corresponding wild type plant cell, i.e. a plant cell which genome has not been modified as per the method.

The abiotic stress tolerance preferably comprises tolerance to salt and/or drought.

The genome of the plant is preferably stably modified such that the modifications are inherited if the plant cell is propagated.

The decreased or inactivated expression may comprise a decreased level or concentration of the protein in the plant cell, a decreased activity of the protein that is present in the plant cell, or a complete absence of the protein in the plant cell.

Accordingly, the genome of the plant cell may be modified such that the decreased or inactivated expression is be obtained at the gene level, e.g., by removing or altering the gene so as to affect the abundance and function of the protein produced. Additionally, or alternatively, the decreased or inactivated expression may be provided in the transcription stage, e.g., by modifying the gene so as to decrease the probability that the gene is transcribed to form mRNA. Additionally, the decreased or inactivated expression may be provided at the mRNA stage by decreasing the likelihood that mRNA is ever translated into the protein, e.g., translational control, or by causing the mRNA to degrade fast.

The decreased or inactivated expression of the protein may for example be obtained by gene silencing, RNA interference (RNAi), virus-induced gene silencing (VIGS), small RNA-mediated post-transcriptional gene silencing, transcription activator-like effector nuclease (TALEN) gene editing techniques, clustered Regularly Interspaced Short Palindromic Repeat (CRISPR/Cas9) gene editing techniques, and/or zinc-finger nuclease (ZFN) gene editing techniques.

The protein (including signal peptide) comprises less than 200 amino acids, such as less than 150 amino acids. Alternatively, the mature protein comprises less than 200 amino acids, such as less than 150 amino acids, such as less than 100 amino acids.

The protein has an amino acid sequence in which at least one part of the amino acid sequence has at least 78%, such as at least 85%, more preferably at least 90% sequence identity to SEQ ID NO: 5. Even more preferably, the at least one part of the amino acid sequence has at least 95, such as at least 99, such as 100% sequence identity to SEQ ID NO: 5. Thus the protein preferably has an amino acid sequence in which at least one part of the amino acid sequence comprises SEQ ID NO: 5.

Preferably the protein has an amino acid sequence in which at least one part of the amino acid sequence has at least 70%, preferably at least 80%, more preferably at least 90% sequence identity to SEQ ID NO: 13, and/or
wherein the protein has an amino acid sequence having at least 80%, more preferably at least 90% coverage to SEQ ID NO: 13.

Preferably the protein has an amino acid sequence in which at least one part of the amino acid sequence has at least 70%, preferably at least 80%, more preferably at least 90% sequence identity to SEQ ID NO: 13, and the protein has an amino acid sequence having at least 80%, more preferably at least 90% coverage to SEQ ID NO: 13.

Accordingly, further studies by the present inventors, as detailed in Example 3Bis, has resulted in the definition of an extended common motif defined in the 72 protein sequence of Solanum tuberosum:

(SEQ ID NO: 13)
EKKDPNVSGVLAKVLASKRRKEAMKESIAKLREKGKPV

As an example, the 72 protein in Nicotiana Benthamiana contains a sequence having a very high similarity to the extended common motif:

(SEQ ID NO: 14, Nicotiana Benthamiana)
EKKDPNMSGVMAKVLASKRRKEAMKESIAKLREKGKPV

Here, SEQ ID NO: 14 has 94.74% sequence identity and 100% coverage to SEQ ID NO: 13.

With reference to all aspects of the technology proposed herein,
and as an alternative to modifying the genome of the plant cell so as to obtain a decreased or inactivated expression of a protein having an amino acid sequence in which at least one part of the amino acid sequence has at least 78%, such as at least 85%, more preferably at least 90% sequence identity to SEQ ID NO: 5,
the genome of the plant cell may instead be modified so as to obtain a decreased or inactivated expression of a protein having an amino acid sequence in which at least one part of the amino acid sequence has at least 70%, preferably at least 80%, more preferably at least 90% sequence identity to SEQ ID NO: 13, and at least 80%, more preferably at least 90% coverage to SEQ ID NO: 13.

Preferably, the protein, in mature form, has an amino acid sequence with at least 30%, such as at least 40%, more preferably at least 47% sequence identity to SEQ ID NO: 1 or to SEQ ID NO: 3. As noted above, SEQ ID NO:1 is the amino acid sequence of the mature 72 protein in Nicotiana benthamiana, whereas SEQ ID NO: 3 is the amino acid sequence of the 72 protein in Solanum tuberosum.

The protein may further be selected among the accessions in table 1 and/or table 2, preferably among the proteins having less than 200 amino acids, or fewer as discussed above.

Sequence identity, also known as identity, is determined as known in the art by comparing sequences (DNA or amino acid), preferably using BLAST (Basic Local Alignment Search Tool) which can be accessed at the ncbi webpage https://blast.ncbi.nlm.nih.qov/Blast.cgi

Coverage, also known as query cover or query coverage, is a number that describes how much of the query sequence is covered by the target sequence. % coverage is the percentage of the query sequence length that is included in the alignment. If the target sequence in the database spans the whole query sequence, then the query cover is 100%.

Preferably, step (b) of modifying the genome of said plant cell comprises modifying the genome so as to fully or partially decrease or inactivate the expression of a gene sequence in said genome, said gene sequence coding for said protein and preferably having at least 90%, such as at least 95%, more preferably at least 99% sequence identity to SEQ ID NO: 6 or SEQ ID NO: 7.

Correspondingly, the expression of the protein in the plant cell is preferably decreased or inactivated by fully or partially decreasing or inactivating the expression of a gene sequence in said genome, said gene sequence coding for said protein and preferably having at least 90%, such as at least 95%, more preferably at least 99% sequence identity to SEQ ID NO: 6 or SEQ ID NO: 7.

Here, SEQ ID NO: 6 corresponds to the Nicotiana benthamiana genomic DNA sequence of gene 72 in Nicotiana benthamiana. SEQ ID NO: 7 in turn corresponds to the 339 nt Nicotiana benthamiana open reading frame sequence coding for the 72 protein.

Preferably, step (b) of modifying the genome of the plant cell comprises mutating or excising at least a part of said gene sequence, preferably using CRISPR/Cas9.

Correspondingly, the genome of the plant cell has preferably been modified by mutating or excising at least a part of said gene sequence, preferably using CRISPR/Cas9.

This is advantageous as it allows the excision of the gene coding for the 72 protein, and thereby provides a complete inactivation of this gene and cessation of the expression of the 72 protein.

As noted above, the decreased or inactivated expression of the protein provides the plant cell with an increased production and concentration of reactive oxygen species (ROS).

Accordingly, the method provides resistance to pathogens that are linked to stress activation of ROS. The method further provides abiotic stress tolerance.

Preferably, the plant cell is selected from the group consisting of a monocot and dicot cell, or the plant cell is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, sugar beet, grape, Arabidopsis and safflower cell, and wherein preferably the plant cell is from the family Solanaceae, preferably from the genus Solanum, more preferably from the species Solanum tuberosum.

This is advantageous in that such plant cells have increased resistance towards Phytophthora infestans, which is responsible for significant economic losses in potato farming. The plant cell and the plant may be selected among the plants mentioned in table 1 and/or table 2.

The increased pathogen resistance may comprise decreased incidence or extent of plant tissue damage, such as leaf lesions, on a plant comprising the plant cell.

Typically, the plant cell has increased resistance towards infection by Phytophthora infestans, compared to a wild type plant cell, the increased resistance comprising a decreased incidence or extent of leaf lesions on a plant comprising the plant cell.

As noted in the examples, infection by Phytophthora infestans leads to leaf lesions. As further noted in the examples, the increased resistance towards infection by Phytophthora infestans is inter alia manifested by reduced incidence and extent of leaf lesions.

Preferably the increased pathogen resistance comprises increased resistance to at least one pathogen selected from the group consisting of Phytophthora infestans, Dickeya dadantii, and Alternaria solani, and the abiotic stress tolerance comprises tolerance towards at least one abiotic stress selected from the group consisting of salt and drought.

Preferably the plant cell has increased pathogen resistance and abiotic stress tolerance. Alternatively, the plant cell has increased pathogen resistance or abiotic stress tolerance.

At least one of the abovementioned objects, or at least one of the further objects which will become evident from the below description, is according to a third aspect of the technology proposed herein further obtained by a method of obtaining a plant having increased pathogen resistance and/or abiotic stress tolerance, the method comprising the steps of
a) performing the method according to any of the preceding claims to obtain a plant cell having increased pathogen resistance and/or abiotic stress tolerance, and
b) cultivating said plant cell to obtain said plant.

Once a plant cell having increased resistance has been obtained, it can be cultivated as known in the art to obtain a plant. The finished plant can then in turn be propagated and/or cultivated to obtain further plants for planting and farming.

At least one of the abovementioned objects, or at least one of the further objects which will become evident from the below description, is according to a fourth aspect of the technology proposed herein further obtained by a plant obtained according to the method of the third aspect of the technology proposed herein.

Correspondingly, a fifth aspect of the technology proposed herein concerns a plant comprising or consisting of one or more plant cells according to the second aspect of the technology proposed herein. The plant is not exclusively obtained by means of an essentially biological process.

The plant is preferably selected from the group consisting of a monocot and dicot plants, or the plant is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, sugar beet, grape, Arabidopsis and safflower cell, and wherein preferably the plant is from the family Solanaceae, preferably from the genus Solanum, more preferably from the species Solanum tuberosum.

A sixth aspect, corresponding to the first and second aspects, of the technology proposed herein concerns the use of the decrease or inactivation of the expression of a protein in a plant cell, wherein the protein has an amino acid sequence in which at least one part of the amino acid sequence has at least 78%, such as at least 85%, more preferably at least 90% sequence identity to SEQ ID NO: 5, and wherein the protein comprises less than 200 amino acids, for providing increased pathogen resistance and/or abiotic stress tolerance to the plant cell.

A seventh aspect of the technology proposed herein concerns progeny of a plant according to the fourth or fifth aspect of the technology proposed herein.

An eight aspect of the technology proposed herein concerns a seed obtained from a plant according to the fourth or fifth aspect of the technology proposed herein.

A ninth aspect of the technology proposed herein concerns a cutting or graft of a plant according to the fourth or fifth aspect of the technology proposed herein.

A tenth aspect of the technology proposed herein concerns a callus of a plant according to the fourth or fifth aspect of the technology proposed herein.

An alternative first aspect of the technology proposed herein concerns a method of obtaining a plant cell having increased stress tolerance, such as increased salt tolerance, the method comprising the steps of
a) providing a plant cell, and
b) modifying the genome of said plant cell so as to obtain a decreased or inactivated expression of a protein having an amino acid sequence in which at least one part of the amino acid sequence has at least 78%, such as at least 85%, more preferably at least 90% sequence identity to SEQ ID NO: 5, and wherein the protein comprises less than 200 amino acids.

### BRIEF DESCRIPTION OF THE DRAWINGS AND DETAILED DESCRIPTION

A more complete understanding of the abovementioned and other features and advantages of the technology proposed herein will be apparent from the following detailed description of preferred embodiments in conjunction with the appended drawings, wherein:

FIG. 1A shows a photograph of a leaf from N. benthamiana with lesions in two sites A and B.

FIG. 1B shows a graph of the mean lesion diameter for the respective sites A and B of the leaf in FIG. 1A.

FIG. 2A shows a photograph of a leaf from a N. benthamiana plant in which the expression of the 72 protein has been silenced.

FIG. 2B shows a photograph of a leaf from a wild type N. benthamiana plant in which the expression of the 72 protein has not been silenced.

FIG. 2C shows a graph of the mean lesion diameter for the respective gene-silenced and wild type plant leaves in FIGS. 2A-2C.

FIG. 2D shows a graph of the mean sporangia per ml obtained for the respective gene-silenced and wild type plant leaves in FIGS. 2A-2C.

FIG. 3A shows a graph of ROS measurement results on wild type vs 72 protein overexpressing plants.

FIG. 3B shows a graph of ROS measurement results on wild type vs 72 protein silenced plants.

FIG. 3C shows a graph of the cumulative results from FIG. 3A.

FIG. 3D shows a graph of the cumulative results from FIG. 3B.

FIG. 4A shows a graph of ROS measurement results of Arabidopsis thaliana plants with silenced expression of the 72 protein vs wild type plants.

FIG. 4B shows a graph of ROS measurement results of Solanum tuberosum plants with silenced expression of the 72 protein vs wild type plants.

FIG. 5 shows P. infestans scoring in field trials of S. tuberosum plants in which the expression of the 72 protein has been silenced.

FIG. 6A shows a photograph of a leaf from a wild type Desiree S. tuberosum plant.

FIG. 6B shows a photograph of a leaf from a S. tuberosum plant in which the expression of the 72 protein has been silenced.

In the figures and the description, the same reference numeral is used to refer to the same feature. One or more ′ added to a reference numeral indicates that the feature so referenced has a similar function, structure or significance as the feature carrying the reference numeral without the one or more ′, however not being identical with this feature.

### EXAMPLE 1

### Inactivation of the 72 Protein in Nicotiana benthamiana Plants Increases Resistance to Late Blight Disease Caused by Phytophthora infestans

### 1.1 Materials and Methods

Nicotiana benthamiana plants were grown in a controlled environmental chamber at the Biotron facility at SLU, Alnarp, Sweden, at 20° C. with a 14/10 hour light/dark cycle. Light intensity was kept at 160 μmol·m⁻²·s⁻¹, and humidity at 65%. Two weeks after seedling transplant, plantlets were put into individual pots and grown for 2-3 weeks.

### 1.1.1 Agrobacterium-mediated Transient Expression

Full-length 72 was PCR amplified from Nicotiana benthamiana cDNA using gene-specific primers containing Gateway attB recombination sites (forward 5′ ggggacaagtttgtacaaaaaagcaggctatggctcggtcattgtctcc and reverse 5′ gccagtcaaagaaccatctcaatagacccagctttcttgtacaaagtggtcccc) SEQ ID NO: 8 and 9). The PCR product was purified and, using BP Clonase, recombined into pDONR201 to generate a pENTRY clone (Invitrogen). The pENTRY insert was recombined into the Gateway plant binary destination vector pK2GW7 using LR clonase.

Agro infiltration was performed using the Agrobacterium-mediated transient expression as described in [1]. A. tumefaciens strain GV2260 harboring pK2GW7 containing 72 or empty pK2GW7 vector were grown in LB liquid medium supplied with antibiotics at 28° C. overnight. Bacteria were pelleted by centrifugation, followed by re-suspension in an infiltration buffer (10 mM MES, 10 mM MgCl2, and 150 μM acetosyringone), at an OD600 of 0.1-0.2. Re-suspended bacterial suspensions were incubated at room temperature in dark condition for 2 hours. Four to five weeks old N. benthamiana leaves were infiltrated using a 1 ml needle-less syringe.

1.1.2 Virus-induced Gene Silencing in Nicotiana benthamiana

Virus-induced gene silencing (VIGS) was carried out in N. benthamiana, as described in [2]. A 72 DNA fragment of approximately 300 bp was designed using the VIGS tool (https://vigs.solgenomics.net/). The fragment was amplified with PCR using forward 5′ ggctacggtctccattcttgagatggttctttgactgg and reverse 5′ tggagacaatgaccgagcggatcgagaccgtagcc primers (SEQ ID NO 10 and 11) compatible with golden gate cloning [3], and cloned into Tobacco Rattle Virus (TRV) RNA2 vector pJK037 using Bsal and DNA ligase [3]. A. tumefaciens strain GV2260 carrying binary plasmid TRV2: 72 or TRV2:GFP were mixed into a 1:1 ratio with a bacteria-containing binary plasmid TRV1 at final OD₆₀₀=0.5 in infiltration buffer. 2-3 weeks old N. benthamiana plants were infiltrated and grown for another 3 weeks in a controlled growth chamber.

1.1.3 Phytophthora infestans Infection Assay

Phytophthora infestans strain 88069 was used for infection studies, as described in [5] with minor modifications. P. infestans was cultured on rye agar medium plates. Two weeks old cultures of P. infestans was used to harvest sporangia. Plates were flooded with water and scraped with L-shaped spreader to release sporangia. Sporangia were filtered through a 40 μm nylon cell strainer to remove hyphae and the sporangia counted using a hemocytometer. The concentration was adjusted to 40000 sporangia per milliliter for VIGS plants infection and 60000 sporangia per milliliter for Agroinfiltrated N. benthamiana leaves.

For overexpression studies, 10 μl of P. infestans sporangia was placed on N. benthamiana leaves infiltrated with Agrobacterium containing pK2GW7:empty or pK2GW7:72 24 h earlier. Plants were kept in a transparent square box supplied with water underneath to maintain 90-100% relative humidity. For VIGS infection studies infiltrated with VIGS constructs 3 weeks earlier were used. 10 μl of sporangia was placed on the detached leafs abaxial side and placed in a box with moist tissue beneath and sealed with parafilm. Data was collected 7 days after infection and data was presented as mean lesion diameter.

### 1.2 Results and Discussion

1.2.1 Agrobacterium-mediated Transient Expression Increases Susceptibility to P. infestans

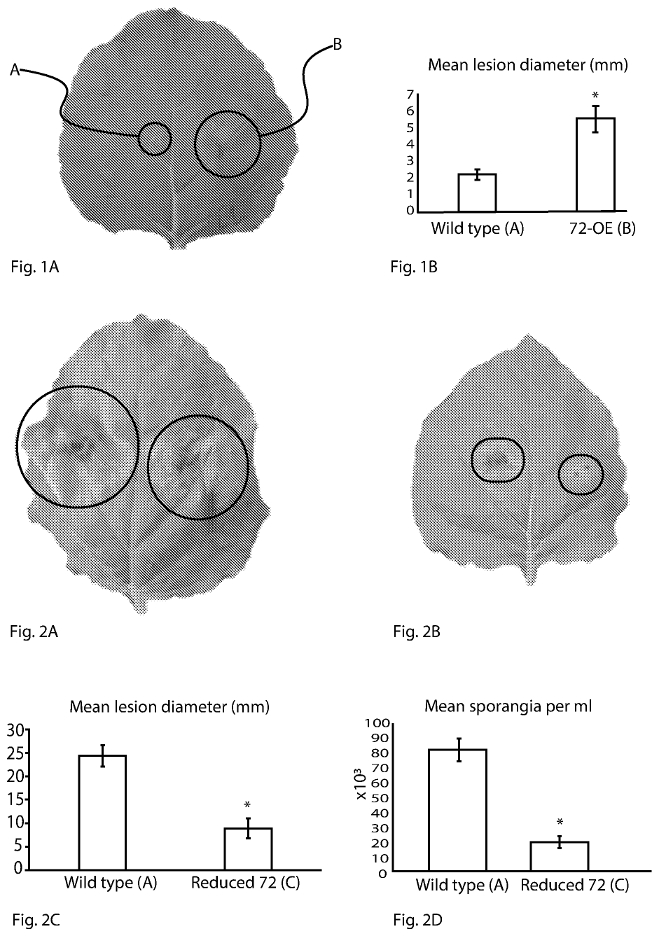

FIG. 1A shows a photograph of a leaf from N. benthamiana. Two lesions, i.e., sites of P. infestans infection, are marked and references as A and B. In site A, agro infiltration was performed using A. tumefaciens strain GV2260 harboring the empty vector, i.e., here there was no transient overexpression of the 72 protein. In contrast, in site B, agro infiltration was performed using A. tumefaciens strain GV2260 harboring the vector coding for the 72 protein. As readily apparent from the photograph, the size of the lesion at site B is significantly larger than at site A. The transient overexpression of the 72 protein in the leaf tissue has thus made the leaf tissue more susceptible, i.e., less resistant, to infection by the pathogen P. infestans.

FIG. 1B shows a bar chart of the corresponding difference in lesion diameter, showing a statistically significant smaller lesion diameter, as highlighted by the * sign, for site A (wild type) where there was no transient overexpression of the 72 protein, compared to site B where the 72 protein was overexpressed (72-OE).

1.2.2 Virus-induced Gene Silencing in Nicotiana benthamiana Improved Resistance to P. infestans

FIG. 2A and 2B shows leaves obtained from wild type Nicotiana benthamiana plants (FIG. 2A) or gene-silenced Nicotiana benthamiana plants (FIG. 2B) 7 days after infection with 10 μl of sporangia from P. infestans. Lesions have formed in both FIG. 2A and FIG. 2B, however, as clearly visible and also circled, the leaf from the plant in which the expression of the 72 protein was silenced has much smaller lesions (FIG. 2B) than the leaf from the wild type plant (FIG. 2A). Accordingly, decreasing the expression of the 72 protein provided the leaf with increased resistance to P. infestans.

### EXAMPLE 2

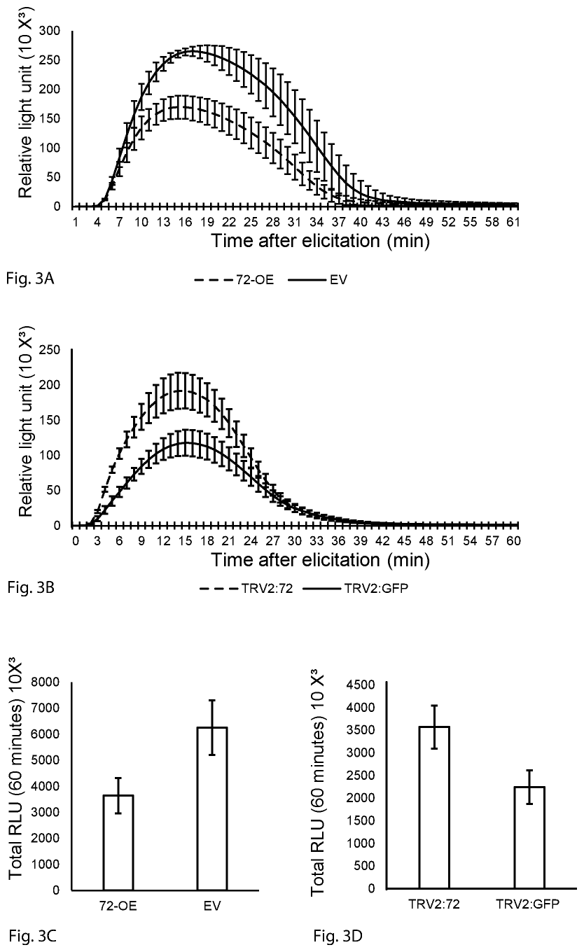

### The Increased Resistance to Infection by P. infestans is not Pathogen-specific as it is Obtained via Increased Abundance of Reactive Oxygen Species (ROS) After Induction by the Immunity-activating Peptide Flagellin (flg22, SEQ ID NO 12). FIG. 3

### 2.1 Materials and Methods

Reactive oxygen species (ROS) was measured from Nicotiana benthamiana leaf discs, as previously described in [4], with minor modifications. Briefly, leaf discs (0.125 cm2 area) were collected from leaves pre-infiltrated with Agrobacterium (24 hpi) or from VIGS plants. Leaf disks were washed with water and placed overnight in 96 well plates with 200 μl water in dark conditions to reduce the effect of tissue damage. Water was replaced with a 200 ul solution containing Luminol (17 mg/ml) and Horseradish peroxidase (10 mg/ml) with 1 μm synthetic flg22 peptide (QRLSTGSRINSAKDDAAGLQIA, SEQ ID NO:12) dissolved in sterile water. ROS production was measured (over 60 minutes) with GloMax® Navigator Microplate Luminometer. ROS burst measured as emitted light due to the oxidation of luminol and represented in a relative light unit (RLU). Data was exported and analyzed in Microsoft excel.

### 2.2 Results and Discussion

The ROS measurements initially showed, see FIGS. 3A and 3C, that Nicotiana benthamiana leaf discs from plants in which the 72 protein had been overexpressed (72-OE), had lower concentration of ROS compared to the control leaf discs after flg22 treatment.

Accordingly, when leaves in which the 72 protein was overexpressed were exposed to the immunity activating flg22 flagellin peptide, lower concentrations of ROS were detected than when the wildtype control leaves were similarly exposed.

The ROS measurements further showed that Nicotiana benthamiana leaf discs from plants in which the expression of the 72 protein had been silenced (Virus-induced gene silencing), see FIG. 3B and FIG. 3D, had higher concentration of ROS compared to the control leaf discs after flg22 treatment.

Accordingly, the decrease or inactivation of the expression of the 72 protein brings about an increased production and concentration of ROS after flg22 treatment, while the overexpression of 72 protein leads to a decreased production and concentration of ROS. Coupled with the observed increased resistance towards infection by P. infestans, it can be concluded that the increased concentration of ROS coincided with the increased resistance. As it is common that plants produce ROS in response to pathogen attack, i.e., as a defense against the attack, then the increased ROS production observed in these results provided a stronger resistance to the pathogens.

Further, it was noted that the immunity activating peptide used was not specific to P. infestans, rather it was a synthetic flagellin peptide, thereby showing that the ROS production and pathogen resistance is not only connected to P. infestans, but instead applicable to a wide variety of pathogens.

### EXAMPLE 3

### A Wide Variety of Plants Have Genes Coding for Plant Specific Variant of the 72 Protein

### 2.1 Materials and Methods

A first BLAST search was run at the ncbi webpage https://blast.ncbi.nlm.nih.gov/Blast.cgi using BLASTP 2.11.0+ and using SEQ ID NO: 1 as query.

By studying the alignments obtained in the first BLAST, which included alignment to the 72 protein in Solanum tuberosum, a common motif, AKVLASKRRKEAMK (SEQ ID NO: 5) was identified. This motif is found in both the 72 protein from N. benthamiana and S. tuberosum.

A second BLAST search was therefore run as above but using SEQ ID NO: 5 as query.

### 2.2 Results and Discussion

### 2.2.1 BLAST 1

The following results were obtained, see table 1:

TABLE 1
Results of BLAST search 1

| | | Query | Per. | Acc. | |
|---|---|---|---|---|---|
| Accession | Description | Cover | ident | Len | nr |
| XP_019259775.1) | PREDICTED: uncharacterized | 100% | 100.00 | 116 | 1 |
| | protein LOC109237842 | | | | |
| | [Nicotiana attenuata] | | | | |
| XP_009779147.1) | PREDICTED: uncharacterized | 100% | 98.21 | 116 | 2 |
| | protein LOC104228135 | | | | |
| | [Nicotiana sylvestris] | | | | |
| XP_009587876.1) | uncharacterized protein | 82% | 89.13 | 114 | 3 |
| | LOC104085525 [Nicotiana | | | | |
| | tomentosiformis] | | | | |
| RLM87146.1) | uncharacterized protein | 87% | 87.76 | 118 | 4 |
| | C2845_PM04G21990 | | | | |
| | [Panicum miliaceum] | | | | |
| PUZ71551.1) | hypothetical protein | 87% | 87.76 | 127 | 5 |
| | GQ55_2G321800 [Panicum | | | | |
| | hallii var. hallii] | | | | |
| KAA8527234.1) | hypothetical protein | 100% | 76.79 | 130 | 6 |
| | F0562_034669 [Nyssa | | | | |
| | sinensis] | | | | |
| XP_016571877.1) | PREDICTED: uncharacterized | 76% | 88.37 | 135 | 7 |
| | protein LOC107870011 isoform | | | | |
| | X1 [Capsicum annuum] | | | | |
| XP_016571878.1) | PREDICTED: uncharacterized | 76% | 88.37 | 130 | 8 |
| | protein LOC107870011 isoform | | | | |
| | X2 [Capsicum annuum] | | | | |
| XP_025803741.1) | uncharacterized protein | 87% | 85.71 | 127 | 9 |
| | LOC112882805 [Panicum | | | | |
| | hallii] | | | | |
| KAF3613377.1) | putative NipSnap protein | 76% | 88.37 | 126 | 10 |
| | K02D10.1-like [Capsicum | | | | |
| | annuum] | | | | |
| PHT54591.1) | hypothetical protein | 76% | 88.37 | 126 | 11 |
| | CQW23_09053 [Capsicum | | | | |
| | baccatum] | | | | |
| XP_031272857.1) | uncharacterized protein | 100% | 75.00 | 157 | 12 |
| | LOC116131325 [Pistacia vera] | | | | |
| KAF3613363.1) | putative thioredoxin-like 1-1 | 76% | 88.37 | 126 | 13 |
| OAY64974.1) | 60S ribosomal protein L5 | 92% | 78.85 | 218 | 14 |
| | [Ananas comosus] | | | | |
| TVU08917.1) | hypothetical protein | 91% | 74.51 | 118 | 15 |
| | EJB05_42344 [Eragrostis | | | | |
| | curvula] | | | | |
| XP_015061235.1) | uncharacterized protein | 71% | 95.00 | 130 | 16 |
| | LOC107007224 [Solanum | | | | |
| | pennellii] | | | | |
| TMW88364.1) | hypothetical protein | 71% | 95.00 | 123 | 17 |
| | EJD97_018654 [Solanum | | | | |
| | chilense] | | | | |
| XP_004252001.1) | uncharacterized protein | 71% | 95.00 | 130 | 18 |
| | LOC101243677 [Solanum | | | | |
| | lycopersicum] | | | | |
| XP_006343159.1) | PREDICTED: uncharacterized | 71% | 95.00 | 128 | 19 |
| | protein LOC102595503 | | | | |
| | [Solanum tuberosum] | | | | |
| PWZ13781.1) | hypothetical protein | 87% | 81.63 | 121 | 20 |
| | Zm00014a_013020 [Zea mays] | | | | |
| GAV86824.1) | hypothetical protein | 91% | 76.47 | 127 | 21 |
| | CFOL_v3_30250 [Cephalotus | | | | |
| | follicularis] | | | | |
| XP_002462669.1) | uncharacterized protein | 87% | 79.59 | 116 | 22 |
| | LOC8054847 [Sorghum | | | | |
| | bicolor] | | | | |
| KAF3451068.1) | hypothetical protein | 91% | 82.35 | 120 | 23 |
| | FNV43_RR07157 [Rhamnella | | | | |
| | rubrinervis] | | | | |
| XP_011628717.1) | uncharacterized protein | 96% | 70.37 | 90 | 24 |
| | LOC105421757 isoform X2 | | | | |
| | [Amborella trichopoda] | | | | |
| GFQ07377.1) | hypothetical protein | 78% | 84.09 | 119 | 25 |
| | PHJA_002881800 | | | | |
| | [Phtheirospermum japonicum] | | | | |
| XP_011073989.1) | uncharacterized protein | 100% | 80.36 | 127 | 26 |
| | LOC105158809 [Sesamum | | | | |
| | indicum] | | | | |
| XP_018852329.1) | uncharacterized protein | 76% | 83.72 | 13 | 27 |
| | LOC109014350 [Juglans regia] | | | | |
| XP_010069234.1) | PREDICTED: uncharacterized | 78% | 84.09 | 128 | 28 |
| | protein LOC104456191 | | | | |
| | [Eucalyptus grandis] | | | | |
| XP_031123519.1) | uncharacterized protein | 76% | 84.78 | 142 | 29 |
| | LOC116026189 [Ipomoea | | | | |
| | triloba] | | | | |
| KAG0544477.1) | hypothetical protein | 87% | 77.55 | 116 | 30 |
| | BDA96_02G278100 [Sorghum | | | | |
| | bicolor] | | | | |
| XP_012075399.1) | uncharacterized protein | 80% | 82.22 | 141 | 31 |
| | LOC105636679 [Jatropha | | | | |
| | curcas] | | | | |
| XP_019160353.1) | PREDICTED: uncharacterized | 76% | 84.78 | 142 | 32 |
| | protein LOC109156914 | | | | |
| | [lpomoea nil] | | | | |
| XP_020087865.1) | uncharacterized protein | 92% | 80.77 | 121 | 33 |
| | LOC109709906 [Ananas | | | | |
| | comosus] | | | | |
| KAF7824305.1) | DNA translocase FtsK [Senna | 78% | 77.27 | 75 | 34 |
| | tora] | | | | |
| XP_022026688.1) | uncharacterized protein | 87% | 83.67 | 118 | 35 |
| | LOC110927329 [Helianthus | | | | |
| | annuus] | | | | |
| XP_008394280.2) | uncharacterized protein | 76% | 79.07 | 139 | 36 |
| | LOC103456350 [Malus | | | | |
| | domestica] | | | | |
| XP_030462088.1) | uncharacterized protein | 78% | 81.82 | 128 | 37 |
| | LOC115682087 [Syzygium | | | | |
| | oleosum] | | | | |
| CAD1838927.1) | unnamed protein product | 92% | 80.77 | 121 | 38 |
| | [Ananas comosus var. | | | | |
| | bracteatus] | | | | |
| KAF3963360.1) | hypothetical protein | 78% | 77.27 | 128 | 39 |
| | CMV_012247 [Castanea | | | | |
| | mollissima] | | | | |
| GAY44833.1) | hypothetical protein | 78% | 79.55 | 130 | 40 |
| | CUMW_084910 [Citrus unshiu] | | | | |
| XP_006492785.1) | uncharacterized protein | 78% | 79.55 | 130 | 41 |
| | LOC102627820 [Citrus | | | | |
| | sinensis] | | | | |
| PON46694.1) | hypothetical protein | 76% | 86.05 | 125 | 42 |
| | PanWU01x14_249900 | | | | |
| | [Parasponia andersonii] | | | | |
| XP_006442192.1) | uncharacterized protein | 78% | 79.55 | 130 | 43 |
| | LOC18047680 [Citrus | | | | |
| | clementina] | | | | |
| TQD88530.1) | hypothetical protein | 76% | 79.07 | 139 | 44 |
| | C1H46_025950 [Malus | | | | |
| | baccata] | | | | |
| XP_022874944.1) | uncharacterized protein | 78% | 77.27 | 125 | 45 |
| | LOC111393576 [Olea | | | | |
| | europaea var. sylvestris] | | | | |
| XP_015869819.1) | uncharacterized protein | 76% | 81.40 | 132 | 46 |
| | LOC107407098 [Ziziphus | | | | |
| | jujuba] | | | | |
| THU62904.1) | hypothetical protein | 98% | 70.91 | 122 | 47 |
| | C4D60_Mb01t10070 [Musa | | | | |
| | balbisiana] | | | | |
| XP_030939313.1) | uncharacterized protein | 78% | 77.27 | 128 | 48 |
| | LOC115964122 [Quercus | | | | |
| | lobata] | | | | |
| XP_023901266.1) | uncharacterized protein | 78% | 77.27 | 128 | 49 |
| | LOC112013112 [Quercus | | | | |
| | suber] | | | | |
| XP_023755590.1) | uncharacterized protein | 76% | 76.74 | 115 | 50 |
| | LOC111904043 [Lactuca | | | | |
| | sativa] | | | | |
| XP_009393511.1) | PREDICTED: uncharacterized | 98% | 70.91 | 122 | 51 |
| | protein LOC103979178 [Musa | | | | |
| | acuminata subsp. malaccensis] | | | | |
| PWA85630.1) | Synaptojanin [Artemisia annua] | 76% | 79.07 | 151 | 52 |
| CAB3457601.1) | unnamed protein product | 69% | 87.18 | 121 | 53 |
| | [Digitaria exilis] | | | | |
| KAF8780812.1) | hypothetical protein | 69% | 87.18 | 124 | 54 |
| | HU200_000757 [Digitaria exilis] | | | | |
| CAB3453955.1) | unnamed protein product | 69% | 87.18 | 142 | 55 |
| | [Digitaria exilis] | | | | |
| XP_034685931.1) | uncharacterized protein | 78% | 70.45 | 129 | 56 |
| | LOC117914629 [Vitis riparia] | | | | |
| XP_008384124.1) | uncharacterized protein | 89% | 72.00 | 138 | 57 |
| | LOC103446765 [Malus | | | | |
| | domestica] | | | | |
| CAB4107654.1) | unnamed protein product | 76% | 76.74 | 115 | 58 |
| | [Lactuca saligna] | | | | |
| PIM99124.1) | hypothetical protein | 78% | 75.00 | 133 | 59 |
| | CDL12_28384 [Handroanthus | | | | |
| | impetiginosus] | | | | |
| TQD80195.1) | hypothetical protein | 100% | 76.79 | 445 | 60 |
| | C1H46_034249 [Malus | | | | |
| | baccata] | | | | |
| XP_028757216.1) | uncharacterized protein | 80% | 75.56 | 151 | 61 |
| | LOC114716379 isoform X1 | | | | |
| | [Prosopis alba] | | | | |
| XP_028757217.1) | uncharacterized protein | 76% | 76.74 | 147 | 62 |
| | LOC114716379 isoform X2 | | | | |
| | [Prosopis alba] | | | | |
| CAD1838960.1) | unnamed protein product | 92% | 80.77 | 249 | 63 |
| | [Ananas comosus var. | | | | |
| | bracteatus] | | | | |
| KAF8701118.1) | hypothetical protein | 69% | 87.18 | 133 | 64 |
| | HU200_033773 [Digitaria exilis] | | | | |
| RXH74223.1) | hypothetical protein | 76% | 79.07 | 2361 | 65 |
| | DVH24_028944 [Malus | | | | |
| | domestica] | | | | |
| PON83292.1) | hypothetical protein | 76% | 83.72 | 152 | 66 |
| | TorRG33x02_208590 [Trema | | | | |
| | orientale] | | | | |
| XP_004287155.1) | PREDICTED: uncharacterized | 71% | 80.00 | 125 | 67 |
| | protein LOC101296797 | | | | |
| | [Fragaria vesca subsp. vesca] | | | | |
| GEV98732.1) | probable phosphoinositide | 76% | 79.07 | 510 | 68 |
| | phosphatase SAC9 isoform X1 | | | | |
| | [Tanacetum cinerariifolium] | | | | |
| XP_030536614.1) | uncharacterized protein | 69% | 87.18 | 128 | 69 |
| | LOC115745288 [Rhodamnia | | | | |
| | argentea] | | | | |
| XP_021612099.1) | uncharacterized protein | 78% | 77.27 | 128 | 70 |
| | LOC110614755 [Manihot | | | | |
| | esculenta] | | | | |
| XP_002277983.1) | PREDICTED: uncharacterized | 78% | 70.45 | 129 | 71 |
| | protein LOC100252564 [Vitis | | | | |
| | vinifera] | | | | |
| RVW13993.1) | hypothetical protein | 78% | 70.45 | 103 | 72 |
| | CK203_086428 [Vitis vinifera] | | | | |
| KAB1214335.1) | hypothetical protein | 69% | 87.18 | 159 | 73 |
| | CJ030_MR5G000478 [Morella | | | | |
| | rubra] | | | | |
| XP_038680339.1) | uncharacterized protein | 71% | 82.50 | 127 | 74 |
| | LOC119981325 [Tripterygium | | | | |
| | wilfordii] | | | | |
| TEY92009.1) | hypothetical protein | 98% | 76.36 | 118 | 75 |
| | Saspl_002643 [Salvia | | | | |
| | splendens] | | | | |
| PSS34837.1) | Zinc transporter like [Actinidia | 76% | 76.74 | 128 | 76 |
| | chinensis var. chinensis] | | | | |
| GFY82842.1) | hypothetical protein | 76% | 76.74 | 128 | 77 |
| | Acr_02g0010820 [Actinidia | | | | |
| | rufa] | | | | |
| TEY11364.1) | hypothetical protein | 98% | 74.55 | 117 | 78 |
| | Saspl_000054 [Salvia | | | | |
| | splendens] | | | | |
| XP_027165752.1) | uncharacterized protein | 78% | 75.56 | 133 | 79 |
| | LOC113765708 [Coffea | | | | |
| | eugenioides] | | | | |
| KAF7116794.1) | hypothetical protein | 76% | 76.74 | 131 | 80 |
| | RHSIM_RhsimUnG0015300 | | | | |
| | [Rhododendron simsii] | | | | |
| KAE9597230.1) | putative CRIB domain- | 75% | 76.19 | 185 | 81 |
| | containing protein [Lupinus | | | | |
| | albus] | | | | |
| KAF7150159.1) | hypothetical protein | 76% | 76.74 | 127 | 82 |
| | RHSIM_Rhsim02G0085800 | | | | |
| | [Rhododendron simsii] | | | | |
| XP_027117818.1) | uncharacterized protein | 78% | 75.56 | 133 | 83 |
| | LOC113735094 [Coffea | | | | |
| | arabica] | | | | |
| XP_004135338.1) | uncharacterized protein | 75% | 76.19 | 133 | 84 |
| | LOC101217329 [Cucumis | | | | |
| | sativus] | | | | |
| XP_027120127.1) | uncharacterized protein | 78% | 75.56 | 133 | 85 |
| | LOC113737076 [Coffea | | | | |
| | arabica] | | | | |
| CDP08012.1) | unnamed protein product | 78% | 75.56 | 132 | 86 |
| | [Coffea canephora] | | | | |
| XP_022998742.1) | uncharacterized protein | 75% | 73.81 | 130 | 87 |
| | LOC111493312 [Cucurbita | | | | |
| | maxima] | | | | |
| XP_022956419.1) | uncharacterized protein | 75% | 73.81 | 130 | 88 |
| | LOC111458160 [Cucurbita | | | | |
| | moschata] | | | | |
| XP_027165358.1) | uncharacterized protein | 78% | 75.56 | 133 | 89 |
| | LOC113765395 [Coffea | | | | |
| | eugenioides] | | | | |
| RXI05542.1) | hypothetical protein | 89% | 72.00 | 2066 | 90 |
| | DVH24_017584 [Malus | | | | |
| | domestica] | | | | |
| XP_022151991.1) | uncharacterized protein | 69% | 82.05 | 141 | 91 |
| | LOC111019817 [Momordica | | | | |
| | charantia] | | | | |
| XP_038877882.1) | uncharacterized protein | 75% | 76.19 | 133 | 92 |
| | LOC120070100 [Benincasa | | | | |
| | hispida] | | | | |
| XP_023528590.1) | uncharacterized protein | 75% | 73.81 | 130 | 93 |
| | LOC111791441 [Cucurbita | | | | |
| | pepo subsp. pepo] | | | | |
| RRT64870.1) | hypothetical protein | 98% | 69.09 | 122 | 94 |
| | B296_00015311 [Ensete | | | | |
| | ventricosum] | | | | |
| RWW25846.1) | hypothetical protein | 98% | 69.09 | 122 | 95 |
| | GW17_00009797 [Ensete | | | | |
| | ventricosum] | | | | |
| TXG72678.1) | hypothetical protein | 75% | 79.07 | 131 | 96 |
| | EZV62_001257 [Acer | | | | |
| | yangbiense] | | | | |
| XP_009366223.1) | PREDICTED: uncharacterized | 89% | 74.00 | 138 | 97 |
| | protein LOC103956019 [Pyrus × | | | | |
| | bretschneideri] | | | | |
| KAF2298351.1) | hypothetical protein | 100% | 76.79 | 342 | 98 |
| | GH714_022860 [Hevea | | | | |
| | brasiliensis] | | | | |
| XP_034916659.1) | uncharacterized protein | 76% | 74.42 | 137 | 99 |
| | LOC118050417 [Populus alba] | | | | |
| OEL31639.1) | hypothetical protein | 73% | 80.49 | 123 | 100 |
| | BAE44_0007341 | | | | |
| | [Dichanthelium oligosanthes] | | | | |

The 100 results in table 1 show that a wide variety of plants contain protein being similar to the 72 protein in N. benthamiana. The proteins identified by the Accessions thus represent plant specific variants of the 72 protein. More particularly, accession XP_006343159.1 (nr 19) designates a Solanum tuberosum specific variant of the 72 protein with a query coverage of 71% and an identity of 95%. Of the 100 proteins, nr 14, 60, 63, 65, 68, 90 and 98 have lengths above 200 amino acids and may therefore belong to other protein families than the 72 protein.

### 2.2.2 BLAST 2

The following results were obtained, see table 2.

TABLE 2
Results of BLAST search 2

| | | Query | Per. | Acc. | |
|---|---|---|---|---|---|
| Accession | Description | Cover | Ident | Len | nr. |
| XP_027345478.1 | uncharacterized protein | 100% | 100% | 161 | 1 |
| | LOC113857605 [Abrus | | | | |
| | precatorius] | | | | |
| ACU18949.1 | unknown [Glycine max] | 100% | 100% | 160 | 2 |
| XP_003518878.1 | myosin-5 [Glycine max] | 100% | 100% | 160 | 3 |
| RDX71423.1 | hypothetical protein | 100% | 100% | 160 | 4 |
| | CR513_49239 [Mucuna | | | | |
| | pruriens] | | | | |
| KAB1214335.1 | hypothetical protein | 100% | 100% | 159 | 5 |
| | CJ030_MR5G000478 [Morella | | | | |
| | rubra] | | | | |
| TKY59155.1 | myosin-5 protein [Spatholobus | 100% | 100% | 157 | 6 |
| | suberectus] | | | | |
| NP_001236138.1 | uncharacterized protein | 100% | 100% | 155 | 7 |
| | LOC100527185 [Glycine max] | | | | |
| PON83292.1 | hypothetical protein | 100% | 100% | 152 | 8 |
| | TorRG33x02_208590 [Trema | | | | |
| | orientale] | | | | |
| XP_020235928.1 | uncharacterized protein | 100% | 100% | 148 | 9 |
| | LOC109815584 [Cajanus | | | | |
| | cajan] | | | | |
| XP_016189999.1 | uncharacterized protein | 100% | 100% | 144 | 10 |
| | LOC107631163 [Arachis | | | | |
| | ipaensis] | | | | |
| XP_031123519.1 | uncharacterized protein | 100% | 100% | 142 | 11 |
| | LOC116026189 [Ipomoea | | | | |
| | triloba] | | | | |
| XP_019160353.1 | PREDICTED: uncharacterized | 100% | 100% | 142 | 12 |
| | protein LOC109156914 | | | | |
| | [Ipomoea nil] | | | | |
| XP_015956575.1 | uncharacterized protein | 100% | 100% | 138 | 13 |
| | LOC107480887 [Arachis | | | | |
| | duranensis] | | | | |
| XP_016571877.1 | PREDICTED: uncharacterized | 100% | 100% | 135 | 14 |
| | protein LOC107870011 | | | | |
| | isoform X1 [Capsicum | | | | |
| | annuum] | | | | |
| XP_027165752.1 | uncharacterized protein | 100% | 100% | 133 | 15 |
| | LOC113765708 [Coffea | | | | |
| | eugenioides] | | | | |
| XP_027120127.1 | uncharacterized protein | 100% | 100% | 133 | 16 |
| | LOC113737076 [Coffea | | | | |
| | arabica] | | | | |
| XP_027117818.1 | uncharacterized protein | 100% | 100% | 133 | 17 |
| | LOC113735094 [Coffea | | | | |
| | arabica] | | | | |
| XP_027165358.1 | uncharacterized protein | 100% | 100% | 133 | 18 |
| | LOC113765395 [Coffea | | | | |
| | eugenioides] | | | | |
| CDP08012.1 | unnamed protein product | 100% | 100% | 132 | 19 |
| | [Coffea canephora] | | | | |
| XP_015061235.1 | uncharacterized protein | 100% | 100% | 130 | 20 |
| | LOC107007224 [Solanum | | | | |
| | pennellii] | | | | |
| XP_016571878.1 | PREDICTED: uncharacterized | 100% | 100% | 130 | 21 |
| | protein LOC107870011 | | | | |
| | isoform X2 [Capsicum | | | | |
| | annuum] | | | | |
| XP_004252001.1 | uncharacterized protein | 100% | 100% | 130 | 22 |
| | LOC101243677 [Solanum | | | | |
| | lycopersicum] | | | | |
| XP_006343159.1 | PREDICTED: uncharacterized | 100% | 100% | 128 | 23 |
| | protein LOC102595503 | | | | |
| | [Solanum tuberosum] | | | | |
| PUZ71551.1 | hypothetical protein | 100% | 100% | 127 | 24 |
| | GQ55_2G321800 [Panicum | | | | |
| | hallii var. hallii] | | | | |
| XP_025803741.1 | uncharacterized protein | 100% | 100% | 127 | 25 |
| | LOC112882805 [Panicum | | | | |
| | hallii] | | | | |
| PHT54591.1 | hypothetical protein | 100% | 100% | 126 | 26 |
| | CQW23_09053 [Capsicum | | | | |
| | baccatum] | | | | |
| KAF3613363.1 | putative thioredoxin-like 1-1, | 100% | 100% | 126 | 27 |
| | chloroplastic-like [Capsicum | | | | |
| | annuum] | | | | |
| KAF3613377.1 | putative NipSnap protein | 100% | 100% | 126 | 28 |
| | K02D10.1-like [Capsicum | | | | |
| | annuum] | | | | |
| PON46694.1 | hypothetical protein | 100% | 100% | 125 | 29 |
| | PanWU01x14_249900 | | | | |
| | [Parasponia andersonii] | | | | |
| OEL31639.1 | hypothetical protein | 100% | 100% | 123 | 30 |
| | BAE44_0007341 | | | | |
| | [Dichanthelium oligosanthes] | | | | |
| TMW88364.1 | hypothetical protein | 100% | 100% | 123 | 31 |
| | EJD97_018654 [Solanum | | | | |
| | chilense] | | | | |
| XP_004957364.2 | uncharacterized protein | 100% | 100% | 118 | 32 |
| | LOC101765483 [Setaria | | | | |
| | italica] | | | | |
| RLM87146.1 | uncharacterized protein | 100% | 100% | 118 | 33 |
| | C2845_PM04G21990 | | | | |
| | [Panicum miliaceum] | | | | |
| XP_009779147.1 | PREDICTED: uncharacterized | 100% | 100% | 116 | 34 |
| | protein LOC104228135 | | | | |
| | [Nicotiana sylvestris] | | | | |
| XP_019259775.1 | PREDICTED: uncharacterized | 100% | 100% | 116 | 35 |
| | protein LOC109237842 | | | | |
| | [Nicotiana attenuata] | | | | |
| XP_009587876.1 | uncharacterized protein | 100% | 100% | 114 | 36 |
| | LOC104085525 [Nicotiana | | | | |
| | tomentosiformis] | | | | |
| PKA60203.1 | hypothetical protein | 100% | 100% | 94 | 37 |
| | AXF42_Ash008262 [Apostasia | | | | |
| | shenzhenica] | | | | |
| XP_007153725.1 | hypothetical protein | 100% | 92.86% | 156 | 38 |
| | PHAVU_003G059800g | | | | |
| | [Phaseolus vulgaris] | | | | |
| XP_017436647.1 | PREDICTED: uncharacterized | 100% | 92.86% | 154 | 39 |
| | protein LOC108343093 [Vigna | | | | |
| | angularis] | | | | |
| XP_014507926.1 | uncharacterized protein | 100% | 92.86% | 153 | 40 |
| | LOC106767524 [Vigna radiata | | | | |
| | var. radiata] | | | | |
| XP_027920164.1 | uncharacterized protein | 100% | 92.86% | 145 | 41 |
| | LOC114178448 [Vigna | | | | |
| | unguiculata] | | | | |
| KAE9597230.1 | putative CRIB domain- | 100% | 92.86% | 185 | 42 |
| | containing protein [Lupinus | | | | |
| | albus] | | | | |
| RXH74223.1 | hypothetical protein | 100% | 92.86% | 2361 | 43 |
| | DVH24_028944 [Malus | | | | |
| | domestica] | | | | |
| RXI05542.1 | hypothetical protein | 100% | 92.86% | 2066 | 44 |
| | DVH24_017584 [Malus | | | | |
| | domestica] | | | | |
| GEV98732.1 | probable phosphoinositide | 100% | 92.86% | 510 | 45 |
| | phosphatase SAC9 isoform X1 | | | | |
| | [Tanacetum cinerariifolium] | | | | |
| TQD80195.1 | hypothetical protein | 100% | 92.86% | 445 | 46 |
| | C1H46_034249 [Malus | | | | |
| | baccata] | | | | |
| TKS11744.1 | 60S ribosomal protein L5-like | 100% | 92.86% | 415 | 47 |
| | [Populus alba] | | | | |
| KAE9452800.1 | hypothetical protein | 100% | 92.86% | 372 | 48 |
| | C3L33_15293 [Rhododendron | | | | |
| | williamsianum] | | | | |
| KAF2298351.1 | hypothetical protein | 100% | 92.86% | 342 | 49 |
| | GH714_022860 [Hevea | | | | |
| | brasiliensis] | | | | |
| CAD1838960.1 | unnamed protein product | 100% | 92.86% | 249 | 50 |
| | [Ananas comosus var. | | | | |
| | bracteatus] | | | | |
| OAY64974.1 | 60S ribosomal protein L5 | 100% | 92.86% | 218 | 51 |
| | [Ananas comosus] | | | | |
| MQL76174.1 | hypothetical protein [Colocasia | 100% | 92.86% | 179 | 52 |
| | esculenta] | | | | |
| XP_018438767.1 | PREDICTED: uncharacterized | 100% | 92.86% | 171 | 53 |
| | protein LOC108811221 | | | | |
| | [Raphanus sativus] | | | | |
| KAF3488523.1 | hypothetical protein | 100% | 92.86% | 169 | 54 |
| | F2Q69_00054674 [Brassica | | | | |
| | cretica] | | | | |
| XP_013643824.1 | uncharacterized protein | 100% | 92.86% | 169 | 55 |
| | BNAC07G14470D [Brassica | | | | |
| | napus] | | | | |
| XP_013594098.1 | PREDICTED: uncharacterized | 100% | 92.86% | 169 | 56 |
| | protein LOC106302124 | | | | |
| | [Brassica oleracea var. | | | | |
| | oleracea] | | | | |
| XP_031474864.1 | uncharacterized protein | 100% | 92.86% | 162 | 57 |
| | LOC116247055 [Nymphaea | | | | |
| | colorata] | | | | |
| XP_003608092.1 | uncharacterized protein | 100% | 92.86% | 160 | 58 |
| | LOC11446352 [Medicago | | | | |
| | truncatula] | | | | |
| XP_031272857.1 | uncharacterized protein | 100% | 92.86% | 157 | 59 |
| | LOC116131325 [Pistacia vera] | | | | |
| XP_013653400.1 | uncharacterized protein | 100% | 92.86% | 157 | 60 |
| | BNAANNG40570D [Brassica | | | | |
| | napus] | | | | |
| XP_009103293.1 | uncharacterized protein | 100% | 92.86% | 157 | 61 |
| | LOC103829375 [Brassica | | | | |
| | rapa] | | | | |
| RID53734.1 | hypothetical protein | 100% | 92.86% | 157 | 62 |
| | BRARA_G01111 [Brassica | | | | |
| | rapa] | | | | |
| XP_013584120.1 | PREDICTED: uncharacterized | 100% | 92.86% | 154 | 63 |
| | protein LOC106292994 | | | | |
| | [Brassica oleracea var. | | | | |
| | oleracea] | | | | |
| XP_021751811.1 | uncharacterized protein | 100% | 92.86% | 151 | 64 |
| | LOC110717424 | | | | |
| | [Chenopodium quinoa] | | | | |
| KZV48470.1 | hypothetical protein | 100% | 92.86% | 151 | 65 |
| | F511_18276 [Dorcoceras | | | | |
| | hygrometricum] | | | | |
| PWA85630.1 | Synaptojanin [Artemisia | 100% | 92.86% | 151 | 66 |
| | annua] | | | | |
| XP_028757216.1 | uncharacterized protein | 100% | 92.86% | 151 | 67 |
| | LOC114716379 isoform X1 | | | | |
| | [Prosopis alba] | | | | |
| XP_021849285.1 | uncharacterized protein | 100% | 92.86% | 150 | 68 |
| | LOC110788952 [Spinacia | | | | |
| | oleracea] | | | | |
| XP_021774812.1 | uncharacterized protein | 100% | 92.86% | 150 | 69 |
| | LOC110738709 | | | | |
| | [Chenopodium quinoa] | | | | |
| KNA05751.1 | hypothetical protein | 100% | 92.86% | 150 | 70 |
| | SOVF_187540 [Spinacia | | | | |
| | oleracea] | | | | |
| XP_007202886.1 | uncharacterized protein | 100% | 92.86% | 148 | 71 |
| | LOC18770848 [Prunus | | | | |
| | persica] | | | | |
| XP_028757217.1 | uncharacterized protein | 100% | 92.86% | 147 | 72 |
| | LOC114716379 isoform X2 | | | | |
| | [Prosopis alba] | | | | |
| CAB4288900.1 | unnamed protein product | 100% | 92.86% | 146 | 73 |
| | [Prunus armeniaca] | | | | |
| VVA99040.1 | unnamed protein product | 100% | 92.86% | 145 | 74 |
| | [Arabis nemorensis] | | | | |
| CAB4319263.1 | unnamed protein product | 100% | 92.86% | 144 | 75 |
| | [Prunus armeniaca] | | | | |
| XP_021814051.1 | uncharacterized protein | 100% | 92.86% | 144 | 76 |
| | LOC110756881 [Prunus | | | | |
| | avium] | | | | |
| XP_010101364.1 | uncharacterized protein | 100% | 92.86% | 143 | 77 |
| | LOC21403280 [Morus | | | | |
| | notabilis] | | | | |
| CAB3453955.1 | unnamed protein product | 100% | 92.86% | 142 | 78 |
| | [Digitaria exilis] | | | | |
| XP_002524858.1 | uncharacterized protein | 100% | 92.86% | 141 | 79 |
| | LOC8286364 [Ricinus | | | | |
| | communis] | | | | |
| XP_012075399.1 | uncharacterized protein | 100% | 92.86% | 141 | 80 |
| | LOC105636679 [Jatropha | | | | |
| | curcas] | | | | |
| XP_011628716.1 | uncharacterized protein | 100% | 92.86% | 140 | 81 |
| | LOC105421757 isoform X1 | | | | |
| | [Amborella trichopoda] | | | | |
| XP_008394280.2 | uncharacterized protein | 100% | 92.86% | 139 | 82 |
| | LOC103456350 [Malus | | | | |
| | domestica] | | | | |
| TQD88530.1 | hypothetical protein | 100% | 92.86% | 139 | 83 |
| | C1H46_025950 [Malus | | | | |
| | baccata] | | | | |
| KAB2618892.1 | hypothetical protein | 100% | 92.86% | 139 | 84 |
| | D8674_014761 [Pyrus | | | | |
| | ussuriensis × Pyrus communis] | | | | |
| XP_008384124.1 | uncharacterized protein | 100% | 92.86% | 138 | 85 |
| | LOC103446765 [Malus | | | | |
| | domestica] | | | | |
| XP_009366223.1 | PREDICTED: uncharacterized | 100% | 92.86% | 138 | 86 |
| | protein LOC103956019 [Pyrus × | | | | |
| | bretschneideri] | | | | |
| XP_009358394.1 | PREDICTED: uncharacterized | 100% | 92.86% | 138 | 87 |
| | protein LOC103949029 [Pyrus × | | | | |
| | bretschneideri] | | | | |
| KAA3477763.1 | Ferrochelatase [Gossypium | 100% | 92.86% | 138 | 88 |
| | australe] | | | | |
| KAF8082458.1 | hypothetical protein | 100% | 92.86% | 137 | 89 |
| | N665_0824s0006 [Sinapis | | | | |
| | alba] | | | | |
| OWM67875.1 | hypothetical protein | 100% | 92.86% | 137 | 90 |
| | CDL15_Pgr010813 [Punica | | | | |
| | granatum] | | | | |
| XP_034916659.1 | uncharacterized protein | 100% | 92.86% | 137 | 91 |
| | LOC118050417 [Populus alba] | | | | |
| XP_031379811.1 | uncharacterized protein | 100% | 92.86% | 137 | 92 |
| | LOC116195006 [Punica | | | | |
| | granatum] | | | | |
| XP_021912054.1 | uncharacterized protein | 100% | 92.86% | 136 | 93 |
| | LOC110825845 [Carica | | | | |
| | papaya] | | | | |
| GFS45937.1 | hypothetical protein | 100% | 92.86% | 135 | 94 |
| | Acr_00g0099090 [Actinidia | | | | |
| | rufa] | | | | |
| PSS32814.1 | Zinc transporter like [Actinidia | 100% | 92.86% | 135 | 95 |
| | chinensis var. chinensis] | | | | |
| XP_017232742.1 | PREDICTED: uncharacterized | 100% | 92.86% | 135 | 96 |
| | protein LOC108206838 | | | | |
| | [Daucus carota subsp. sativus] | | | | |
| PIM99124.1 | hypothetical protein | 100% | 92.86% | 133 | 97 |
| | CDL12_28384 [Handroanthus | | | | |
| | impetiginosus] | | | | |
| KAF8701118.1 | hypothetical protein | 100% | 92.86% | 133 | 98 |
| | HU200_033773 [Digitaria | | | | |
| | exilis] | | | | |
| TYI60906.1 | hypothetical protein | 100% | 92.86% | 132 | 99 |
| | E1A91_D10G136800v1 | | | | |
| | [Gossypium mustelinum] | | | | |
| MBA0779824.1 | hypothetical protein | 100% | 92.86% | 132 | 100 |
| | [Gossypium trilobum] | | | | |
| MBA0846036.1 | hypothetical protein | 100% | 92.86% | 132 | 101 |
| | [Gossypium armourianum] | | | | |
| MBA0870982.1 | hypothetical protein | 100% | 92.86% | 132 | 102 |
| | [Gossypium schwendimanii] | | | | |
| XP_015869819.1 | uncharacterized protein | 100% | 92.86% | 132 | 103 |
| | LOC107407098 [Ziziphus | | | | |
| | jujuba] | | | | |
| XP_012456073.1 | PREDICTED: uncharacterized | 100% | 92.86% | 132 | 104 |
| | protein LOC105777371 | | | | |
| | [Gossypium raimondii] | | | | |
| XP_016677524.1 | PREDICTED: uncharacterized | 100% | 92.86% | 132 | 105 |
| | protein LOC107896768 | | | | |
| | [Gossypium hirsutum] | | | | |
| MBA0628543.1 | hypothetical protein | 100% | 92.86% | 132 | 106 |
| | [Gossypium davidsonii] | | | | |
| KAB2008928.1 | hypothetical protein | 100% | 92.86% | 132 | 107 |
| | ES319_D10G133400v1 | | | | |
| | [Gossypium barbadense] | | | | |
| MBA0696145.1 | hypothetical protein | 100% | 92.86% | 132 | 108 |
| | [Gossypium aridum] | | | | |
| KAE8076637.1 | hypothetical protein | 100% | 92.86% | 131 | 109 |
| | FH972_015274 [Carpinus | | | | |
| | fangiana] | | | | |
| PIA36149.1 | hypothetical protein | 100% | 92.86% | 131 | 110 |
| | AQUCO_03400216v1 | | | | |
| | [Aquilegia coerulea] | | | | |
| XP_022722981.1 | uncharacterized protein | 100% | 92.86% | 131 | 11 |
| | LOC111280086 [Durio | | | | |
| | zibethinus] | | | | |
| TXG72678.1 | hypothetical protein | 100% | 92.86% | 131 | 112 |
| | EZV62_001257 [Acer | | | | |
| | yangbiense] | | | | |
| CAA7047813.1 | unnamed protein product | 100% | 92.86% | 131 | 113 |
| | [Microthlaspi erraticum] | | | | |
| XP_018852329.1 | uncharacterized protein | 100% | 92.86% | 131 | 114 |
| | LOC109014350 [Juglans | | | | |
| | regia] | | | | |
| KAF7116794.1 | hypothetical protein | 100% | 92.86% | 131 | 115 |
| | RHSIM_RhsimUnG0015300 | | | | |
| | [Rhododendron simsii] | | | | |
| KAF2553190.1 | hypothetical protein | 100% | 92.86% | 131 | 116 |
| | F2Q68_00035361 [Brassica | | | | |
| | cretica] | | | | |
| XP_006492785.1 | uncharacterized protein | 100% | 92.86% | 130 | 117 |
| | LOC102627820 [Citrus | | | | |
| | sinensis] | | | | |
| GAY44833.1 | hypothetical protein | 100% | 92.86% | 130 | 118 |
| | CUMW_084910 [Citrus unshiu] | | | | |
| KAF0914048.1 | hypothetical protein | 100% | 92.86% | 130 | 119 |
| | E2562_026467 [Oryza | | | | |
| | meyeriana var. granulata] | | | | |
| XP_006442192.1 | uncharacterized protein | 100% | 92.86% | 130 | 120 |
| | LOC18047680 [Citrus | | | | |
| | clementina] | | | | |
| KAA8527234.1 | hypothetical protein | 100% | 92.86% | 130 | 121 |
| | F0562_034669 [Nyssa | | | | |
| | sinensis] | | | | |
| KAF9836182.1 | hypothetical protein | 100% | 92.86% | 129 | 122 |
| | H0E87_000267 [Populus | | | | |
| | deltoides] | | | | |
| XP_006376459.1 | uncharacterized protein | 100% | 92.86% | 129 | 123 |
| | LOC18104606 [Populus | | | | |
| | trichocarpa] | | | | |
| XP_002277983.1 | PREDICTED: uncharacterized | 100% | 92.86% | 129 | 124 |
| | protein LOC100252564 [Vitis | | | | |
| | vinifera] | | | | |
| XP_034685931.1 | uncharacterized protein | 100% | 92.86% | 129 | 125 |
| | LOC117914629 [Vitis riparia] | | | | |
| XP_011037360.1 | PREDICTED: uncharacterized | 100% | 92.86% | 129 | 126 |
| | protein LOC105134595 | | | | |
| | [Populus euphratica] | | | | |
| GFY82842.1 | hypothetical protein | 100% | 92.86% | 128 | 127 |
| | Acr_02g0010820 [Actinidia | | | | |
| | rufa] | | | | |
| XP_010069234.1 | PREDICTED: uncharacterized | 100% | 92.86% | 128 | 128 |
| | protein LOC104456191 | | | | |
| | [Eucalyptus grandis] | | | | |
| XP_021612099.1 | uncharacterized protein | 100% | 92.86% | 128 | 129 |
| | LOC110614755 [Manihot | | | | |
| | esculenta] | | | | |
| PSS34837.1 | Zinc transporter like [Actinidia | 100% | 92.86% | 128 | 130 |
| | chinensis var. chinensis] | | | | |
| XP_018439813.1 | PREDICTED: uncharacterized | 100% | 92.86% | 127 | 131 |
| | protein LOC108812136 | | | | |
| | [Raphanus sativus] | | | | |
| XP_010927914.1 | uncharacterized protein | 100% | 92.86% | 127 | 132 |
| | LOC105049841 [Elaeis | | | | |
| | guineensis] | | | | |
| XP_038680339.1 | uncharacterized protein | 100% | 92.86% | 127 | 133 |
| | LOC119981325 [Tripterygium | | | | |
| | wilfordii] | | | | |
| XP_011073989.1 | uncharacterized protein | 100% | 92.86% | 127 | 134 |
| | LOC105158809 [Sesamum | | | | |
| | indicum] | | | | |
| GAV86824.1 | hypothetical protein | 100% | 92.86% | 127 | 135 |
| | CFOL_v3_30250 [Cephalotus | | | | |
| | follicularis] | | | | |
| KAF7150159.1 | hypothetical protein | 100% | 92.86% | 127 | 136 |
| | RHSIM_Rhsim02G0085800 | | | | |
| | [Rhododendron simsii] | | | | |
| XP_021291697.1 | uncharacterized protein | 100% | 92.86% | 126 | 137 |
| | LOC110422204 [Herrania | | | | |
| | umbratica] | | | | |
| XP_022556935.1 | uncharacterized protein | 100% | 92.86% | 125 | 138 |
| | LOC111205422 [Brassica | | | | |
| | napus] | | | | |
| XP_013642341.1 | uncharacterized protein | 100% | 92.86% | 125 | 139 |
| | LOC106347363 [Brassica | | | | |
| | napus] | | | | |
| XP_004287155.1 | PREDICTED: uncharacterized | 100% | 92.86% | 125 | 140 |
| | protein LOC101296797 | | | | |
| | [Fragaria vesca subsp. vesca] | | | | |
| VDC66309.1 | unnamed protein product | 100% | 92.86% | 125 | 141 |
| | [Brassica rapa] | | | | |
| XP_024181017.1 | uncharacterized protein | 100% | 92.86% | 125 | 142 |
| | LOC112186748 [Rosa | | | | |
| | chinensis] | | | | |
| XP_012839055.1 | PREDICTED: uncharacterized | 100% | 92.86% | 125 | 143 |
| | protein LOC105959491 | | | | |
| | [Erythranthe guttata] | | | | |
| VDD43187.1 | unnamed protein product | 100% | 92.86% | 125 | 144 |
| | [Brassica oleracea] | | | | |
| KAF8780812.1 | hypothetical protein | 100% | 92.86% | 124 | 145 |
| | HU200_000757 [Digitaria | | | | |
| | exilis] | | | | |
| RID58239.1 | hypothetical protein | 100% | 92.86% | 123 | 146 |
| | BRARA_F01546 [Brassica | | | | |
| | rapa] | | | | |
| RWW25846.1 | hypothetical protein | 100% | 92.86% | 122 | 147 |
| | GW17_00009797 [Ensete | | | | |
| | ventricosum] | | | | |
| THU62904.1 | hypothetical protein | 100% | 92.86% | 122 | 148 |
| | C4D60_Mb01t10070 [Musa | | | | |
| | balbisiana] | | | | |
| XP_009393511.1 | PREDICTED: uncharacterized | 100% | 92.86% | 122 | 149 |
| | protein LOC103979178 [Musa | | | | |
| | acuminata subsp. | | | | |
| | malaccensis] | | | | |
| KAE8718297.1 | Ribosomal protein L5 B | 100% | 92.86% | 122 | 150 |
| | isoform 1 [Hibiscus syriacus] | | | | |
| RRT64870.1 | hypothetical protein | 100% | 92.86% | 122 | 151 |
| | B296_00015311 [Ensete | | | | |
| | ventricosum] | | | | |
| CAD1838927.1 | unnamed protein product | 100% | 92.86% | 121 | 152 |
| | [Ananas comosus var. | | | | |
| | bracteatus] | | | | |
| XP_020087865.1 | uncharacterized protein | 100% | 92.86% | 121 | 153 |
| | LOC109709906 [Ananas | | | | |
| | comosus] | | | | |
| CAB3457601.1 | unnamed protein product | 100% | 92.86% | 121 | 154 |
| | [Digitaria exilis] | | | | |
| KAF3451068.1 | hypothetical protein | 100% | 92.86% | 120 | 155 |
| | FNV43_RR07157 [Rhamnella | | | | |
| | rubrinervis] | | | | |
| XP_010539694.1 | PREDICTED: uncharacterized | 100% | 92.86% | 120 | 156 |
| | protein LOC104813690 | | | | |
| | [Tarenaya hassleriana] | | | | |
| XP_015612239.1 | uncharacterized protein | 100% | 92.86% | 120 | 157 |
| | LOC4347585 [Oryza sativa | | | | |
| | Japonica Group] | | | | |
| GFQ07377.1 | hypothetical protein | 100% | 92.86% | 119 | 158 |
| | PHJA_002881800 | | | | |
| | [Phtheirospermum japonicum] | | | | |
| TEY92009.1 | hypothetical protein | 100% | 92.86% | 118 | 159 |
| | Saspl_002643 [Salvia | | | | |
| | splendens] | | | | |
| XP_022026688.1 | uncharacterized protein | 100% | 92.86% | 118 | 160 |
| | LOC110927329 [Helianthus | | | | |
| | annuus] | | | | |
| TEY11364.1 | hypothetical protein | 100% | 92.86% | 117 | 161 |
| | Saspl_000054 [Salvia | | | | |
| | splendens] | | | | |
| XP_008807249.1 | uncharacterized protein | 100% | 92.86% | 115 | 162 |
| | LOC103719676 [Phoenix | | | | |
| | dactylifera] | | | | |
| BAD33829.1 | unknown protein [Oryza sativa | 100% | 92.86% | 106 | 163 |
| | Japonica Group] | | | | |
| KAF2531731.1 | hypothetical protein | 100% | 92.86% | 106 | 164 |
| | F2Q70_00030979 [Brassica | | | | |
| | cretica] | | | | |
| RVW13993.1 | hypothetical protein | 100% | 92.86% | 103 | 165 |
| | CK203_086428 [Vitis vinifera] | | | | |
| KAA3457622.1 | 60S ribosomal protein L5 | 100% | 92.86% | 100 | 166 |
| | [Gossypium australe] | | | | |
| XP_011628717.1 | uncharacterized protein | 100% | 92.86% | 90 | 167 |
| | LOC105421757 isoform X2 | | | | |
| | [Amborella trichopoda] | | | | |
| GFS45913.1 | hypothetical protein | 100% | 92.86% | 88 | 168 |
| | Acr_00g0098960 [Actinidia | | | | |
| | rufa] | | | | |
| THF94571.1 | hypothetical protein | 100% | 85.71% | 178 | 169 |
| | TEA_009452 [Camellia | | | | |
| | sinensis var. sinensis] | | | | |
| KAE8663101.1 | Ribosomal protein L5 B | 100% | 85.71% | 172 | 170 |
| | isoform 1 [Hibiscus syriacus] | | | | |
| XP_028102213.1 | uncharacterized protein | 100% | 85.71% | 142 | 171 |
| | LOC114301462 [Camellia | | | | |
| | sinensis] | | | | |
| KAF5950490.1 | hypothetical protein | 100% | 85.71% | 141 | 172 |
| | HYC85_012483 [Camellia | | | | |
| | sinensis] | | | | |
| XP_030536614.1 | uncharacterized protein | 100% | 85.71% | 128 | 173 |
| | LOC115745288 [Rhodamnia | | | | |
| | argentea] | | | | |
| XP_030462088.1 | uncharacterized protein | 100% | 85.71% | 128 | 174 |
| | LOC115682087 [Syzygium | | | | |
| | oleosum] | | | | |
| KMZ66960.1 | hypothetical protein | 100% | 85.71% | 127 | 175 |
| | ZOSMA_27G00050 [Zostera | | | | |
| | marina] | | | | |
| XP_022874944.1 | uncharacterized protein | 100% | 85.71% | 125 | 176 |
| | LOC111393576 [Olea | | | | |
| | europaea var. sylvestris] | | | | |
| XP_004505146.1 | uncharacterized protein | 100% | 85.71% | 158 | 177 |
| | LOC101506653 [Cicer | | | | |
| | arietinum] | | | | |
| XP_019423319.1 | PREDICTED: uncharacterized | 100% | 92.86% | 156 | 178 |
| | protein LOC109332755 | | | | |
| | [Lupinus angustifolius] | | | | |
| PQQ03363.1 | uncharacterized protein | 100% | 85.71% | 148 | 179 |
| | Pyn_01435 [Prunus yedoensis | | | | |
| | var. nudiflora] | | | | |
| PQP96424.1 | uncharacterized protein | 100% | 85.71% | 144 | 180 |
| | Pyn_29056 [Prunus yedoensis | | | | |
| | var. nudiflora] | | | | |
| XP_037435418.1 | uncharacterized protein | 100% | 85.71% | 184 | 181 |
| | LOC119302487 [Triticum | | | | |
| | dicoccoides] | | | | |
| XP_020199146.1 | uncharacterized protein | 100% | 85.71% | 183 | 182 |
| | LOC109784946 [Aegilops | | | | |
| | tauschii subsp. tauschii] | | | | |
| XP_037442698.1 | uncharacterized protein | 100% | 85.71% | 159 | 183 |
| | LOC119311166 [Triticum | | | | |
| | dicoccoides] | | | | |
| BAK07041.1 | predicted protein [Hordeum | 100% | 85.71% | 145 | 184 |
| | vulgare subsp. vulgare] | | | | |
| KAF7073652.1 | hypothetical protein | 100% | 85.71% | 142 | 185 |
| | CFC21_078603 [Triticum | | | | |
| | aestivum] | | | | |
| XP_003576734.1 | uncharacterized protein | 100% | 85.71% | 131 | 186 |
| | LOC100826069 | | | | |
| | [Brachypodium distachyon] | | | | |
| XP_023528590.1 | uncharacterized protein | 100% | 85.71% | 130 | 187 |
| | LOC111791441 [Cucurbita | | | | |
| | pepo subsp. pepo] | | | | |
| XP_022956419.1 | uncharacterized protein | 100% | 85.71% | 130 | 188 |
| | LOC111458160 [Cucurbita | | | | |
| | moschata] | | | | |
| XP_022998742.1 | uncharacterized protein | 100% | 85.71% | 130 | 189 |
| | LOC111493312 [Cucurbita | | | | |
| | maxima] | | | | |
| XP_023901266.1 | uncharacterized protein | 100% | 85.71% | 128 | 190 |
| | LOC112013112 [Quercus | | | | |
| | suber] | | | | |
| KAF3963360.1 | hypothetical protein | 100% | 85.71% | 128 | 191 |
| | CMV_012247 [Castanea | | | | |
| | mollissima] | | | | |
| XP_030939370.1 | uncharacterized protein | 100% | 85.71% | 128 | 192 |
| | LOC115964163 [Quercus | | | | |
| | lobata] | | | | |
| XP_030939313.1 | uncharacterized protein | 100% | 85.71% | 128 | 193 |
| | LOC115964122 [Quercus | | | | |
| | lobata] | | | | |
| XP_009149667.1 | uncharacterized protein | 100% | 85.71% | 125 | 194 |
| | LOC103872987 [Brassica | | | | |
| | rapa] | | | | |
| NP_001142904.1 | uncharacterized protein | 100% | 85.71% | 123 | 195 |
| | LOC100275335 [Zea mays] | | | | |
| PWZ13781.1 | hypothetical protein | 100% | 85.71% | 121 | 196 |
| | Zm00014a_013020 [Zea | | | | |
| | mays] | | | | |
| TVU08917.1 | hypothetical protein | 100% | 85.71% | 118 | 197 |
| | EJB05_42344 [Eragrostis | | | | |
| | curvula] | | | | |
| KAG0544477.1 | hypothetical protein | 100% | 85.71% | 116 | 198 |
| | BDA96_02G278100 [Sorghum | | | | |
| | bicolor] | | | | |
| XP_002462669.1 | uncharacterized protein | 100% | 85.71% | 116 | 199 |
| | LOC8054847 [Sorghum | | | | |
| | bicolor] | | | | |
| EMS51334.1 | hypothetical protein | 100% | 85.71% | 81 | 200 |
| | TRIUR3_03050 [Triticum | | | | |
| | urartu] | | | | |
| EPS63920.1 | hypothetical protein | 92% | 92.31% | 76 | 201 |
| | M569_10863 [Genlisea aurea] | | | | |
| KVH91022.1 | Synaptojanin, N-terminal | 100% | 85.71% | 1996 | 202 |
| | [Cynara cardunculus var. | | | | |
| | scolymus] | | | | |
| XP_024985152.1 | uncharacterized protein | 100% | 85.71% | 147 | 203 |
| | LOC112520804 [Cynara | | | | |
| | cardunculus var. scolymus] | | | | |
| KAF3341042.1 | hypothetical protein | 100% | 85.71% | 134 | 204 |
| | FCM35_KLT09886 [Carex | | | | |
| | littledalei] | | | | |
| NP_564142.1 | hypothetical protein | 100% | 85.71% | 126 | 205 |
| | AT1G21500 [Arabidopsis | | | | |
| | thaliana] | | | | |
| XP_002893169.1 | uncharacterized protein | 100% | 85.71% | 126 | 206 |
| | LOC9329230 [Arabidopsis | | | | |
| | lyrata subsp. lyrata] | | | | |
| OAP12061.1 | hypothetical protein | 100% | 85.71% | 126 | 207 |
| | AXX17_AT1G22560 | | | | |
| | [Arabidopsis thaliana] | | | | |
| XP_010496341.1 | PREDICTED: uncharacterized | 100% | 85.71% | 125 | 208 |
| | protein LOC104773428 | | | | |
| | [Camelina sativa] | | | | |
| XP_010459860.1 | PREDICTED: uncharacterized | 100% | 85.71% | 125 | 209 |
| | protein LOC104740846 | | | | |
| | [Camelina sativa] | | | | |
| XP_010498605.1 | PREDICTED: uncharacterized | 100% | 85.71% | 125 | 210 |
| | protein LOC104776270 | | | | |
| | [Camelina sativa] | | | | |
| XP_010468018.1 | PREDICTED: uncharacterized | 100% | 85.71% | 116 | 211 |
| | protein LOC104748022 | | | | |
| | [Camelina sativa] | | | | |
| CAB4107654.1 | unnamed protein product | 100% | 85.71% | 115 | 212 |
| | [Lactuca saligna] | | | | |
| XP_023755590.1 | uncharacterized protein | 100% | 85.71% | 115 | 213 |
| | LOC111904043 [Lactuca | | | | |
| | sativa] | | | | |
| BAH57254.1 | AT1G21500 [Arabidopsis | 100% | 85.71% | 71 | 214 |
| | thaliana] | | | | |
| KAF4387597.1 | hypothetical protein | 100% | 85.71% | 2024 | 215 |
| | G4B88_003924 [Cannabis | | | | |
| | sativa] | | | | |
| RZC64230.1 | hypothetical protein | 100% | 85.71% | 359 | 216 |
| | C5167_025994 [Papaver | | | | |
| | somniferum] | | | | |
| RZC47730.1 | hypothetical protein | 100% | 85.71% | 310 | 217 |
| | C5167_040675 [Papaver | | | | |
| | somniferum] | | | | |
| XP_030492085.1 | protein FAM170B [Cannabis | 100% | 85.71% | 163 | 218 |
| | sativa] | | | | |
| XP_026439270.1 | uncharacterized protein | 100% | 85.71% | 135 | 219 |
| | LOC113337950 [Papaver | | | | |
| | somniferum] | | | | |
| XP_026383206.1 | uncharacterized protein | 100% | 85.71% | 135 | 220 |
| | LOC113278618 [Papaver | | | | |
| | somniferum] | | | | |
| KAF6137476.1 | hypothetical protein | 100% | 85.71% | 132 | 221 |
| | GIB67_027863 [Kingdonia | | | | |
| | uniflora] | | | | |
| EOY04625.1 | Uncharacterized protein | 100% | 85.71% | 126 | 222 |
| | TCM_019839 [Theobroma | | | | |
| | cacao] | | | | |
| XP_007033699.2 | PREDICTED: uncharacterized | 100% | 85.71% | 126 | 223 |
| | protein LOC18602328 | | | | |
| | [Theobroma cacao] | | | | |
| KAF4352531.1 | hypothetical protein | 100% | 85.71% | 126 | 224 |
| | G4B88_013361 [Cannabis | | | | |
| | sativa] | | | | |
| KAF2325560.1 | hypothetical protein | 100% | 85.71% | 355 | 225 |
| | GH714_030366 [Hevea | | | | |
| | brasiliensis] | | | | |
| KAF2325557.1 | hypothetical protein | 100% | 85.71% | 334 | 226 |
| | GH714_030328 [Hevea | | | | |
| | brasiliensis] | | | | |
| GAU14550.1 | hypothetical protein | 100% | 85.71% | 163 | 227 |
| | TSUD_96140 [Trifolium | | | | |
| | subterraneum] | | | | |
| PNY16259.1 | hypothetical protein | 100% | 85.71% | 161 | 228 |
| | L195_g012974 [Trifolium | | | | |
| | pratense] | | | | |
| XP_008243041.1 | PREDICTED: uncharacterized | 100% | 85.71% | 146 | 229 |
| | protein LOC103341312 | | | | |
| | [Prunus mume] | | | | |
| XP_022151991.1 | uncharacterized protein | 100% | 85.71% | 141 | 230 |
| | LOC111019817 [Momordica | | | | |
| | charantia] | | | | |
| XP_004135338.1 | uncharacterized protein | 100% | 85.71% | 133 | 231 |
| | LOC101217329 [Cucumis | | | | |
| | sativus] | | | | |
| XP_008446014.1 | PREDICTED: uncharacterized | 100% | 85.71% | 133 | 232 |
| | protein LOC103488868 | | | | |
| | [Cucumis melo] | | | | |
| XP_038877882.1 | uncharacterized protein | 100% | 85.71% | 133 | 233 |
| | LOC120070100 [Benincasa | | | | |
| | hispida] | | | | |
| KAF9606348.1 | hypothetical protein | 100% | 85.71% | 132 | 234 |
| | IFM89_025016 [Coptis | | | | |
| | chinensis] | | | | |
| XP_021638265.1 | uncharacterized protein | 100% | 85.71% | 125 | 235 |
| | LOC110633804 [Hevea | | | | |
| | brasiliensis] | | | | |
| TYK15742.1 | Lipase maturation factor 2 | 100% | 85.71% | 113 | 236 |
| | [Cucumis melo var. makuwa] | | | | |
| KAF7824305.1 | DNA translocase FtsK [Senna | 100% | 85.71% | 75 | 237 |
| | tora] | | | | |
| XP_010672453.1 | PREDICTED: uncharacterized | 100% | 78.57% | 157 | 238 |
| | protein LOC104889021 [Beta | | | | |
| | vulgaris subsp. vulgaris] | | | | |
| KFK44247.1 | hypothetical protein | 100% | 78.57% | 154 | 239 |
| | AALP_AA1G233900 [Arabis | | | | |
| | alpina] | | | | |
| XP_020248015.1 | uncharacterized protein | 100% | 78.57% | 107 | 240 |
| | LOC109825570 [Asparagus | | | | |
| | officinalis] | | | | |
| RWR71906.1 | DNA translocase FtsK | 92% | 84.62% | 132 | 241 |
| | [Cinnamomum micranthum f. | | | | |
| | kanehirae] | | | | |
| OVA05160.1 | hypothetical protein | 100% | 85.71% | 135 | 242 |
| | BVC80_8895g28 [Macleaya | | | | |
| | cordata] | | | | |
| XP_010258445.1 | PREDICTED: uncharacterized | 100% | 85.71% | 128 | 243 |
| | protein LOC104598219 | | | | |
| | [Nelumbo nucifera] | | | | |
| DAD43393.1 | TPA: hypothetical protein | 100% | 85.71% | 70 | 244 |
| | HUJ06_001623 [Nelumbo | | | | |
| | nucifera] | | | | |
| KAF5187773.1 | hypothetical protein | 100% | 78.57% | 135 | 245 |
| | FRX31_022643 [Thalictrum | | | | |
| | thalictroides] | | | | |
| XP_006416295.1 | uncharacterized protein | 100% | 78.57% | 136 | 246 |
| | LOC18992793 [Eutrema | | | | |
| | salsugineum] | | | | |
| XP_006304297.1 | uncharacterized protein | 100% | 78.57% | 129 | 247 |
| | LOC17900587 [Capsella | | | | |
| | rubella] | | | | |
| XP_006661439.2 | PREDICTED: uncharacterized | 100% | 78.57% | 172 | 248 |
| | protein LOC102701106 [Oryza | | | | |
| | brachyantha] | | | | |
| GFP82600.1 | hypothetical protein | 92% | 76.92% | 79 | 249 |
| | PHJA_000403100 | | | | |
| | [Phtheirospermum japonicum] | | | | |
| MBC9844332.1 | hypothetical protein [Adiantum | 100% | 78.57% | 172 | 250 |
| | capillus-veneris] | | | | |
| XP_020587853.1 | uncharacterized protein | 100% | 78.57% | 101 | 251 |
| | LOC110029765 [Phalaenopsis | | | | |
| | equestris] | | | | |
| XP_020699488.1 | uncharacterized protein | 100% | 78.57% | 100 | 252 |
| | LOC110111810 [Dendrobium | | | | |
| | catenatum] | | | | |

The results in table 2 show 252 proteins having more that 78% identity to the motif AKVLASKRRKEAMK (SEQ ID NO: 5). The results include the cereals. Of the 252 protein, 15 (nr 43-51, 202, 2015-217, 225-226) have lengths above 200 amino acids and may therefore belong to other protein families than the 72 protein. The remaining proteins represent plant specific variants of the 72 protein.

### EXAMPLE 3Bis

### Definition of an Extended Common Motif

The results of Example 3 were further studied by the present inventors resulting in an extended common motif which in Solanum tuberosum has the sequence:

(SEQ ID NO: 13, Solanum tuberosum)
EKKDPNVSGVLAKVLASKRRKEAMKESIAKLREKGKPV

This extended common motif is 38 amino acids long, and thus covers 38/55 (69%) of the amino acids in the mature 72 protein in Solanum tuberosum:

(SEQ ID NO: 3)
AARRPPPPPPTEKKDPNVSGVLAKVLASKRRKEAMKESIAKLREKGKPV
KEVPSE

The extended common motif (SEQ ID NO: 13) further has a 94.74% sequence identity and 100% coverage to the corresponding sequence in Nicotiana benthamiana (SEQ ID NO: 14):

(SEQ ID NO: 14, Nicotiana benthamiana)
EKKDPNMSGVMAKVLASKRRKEAMKESIAKLREKGKPV

The corresponding sequence in Nicotiana benthamiana is also 38 amino acids long and covers 38/56 (68%) of the amino acids in the mature 72 protein in Nicotiana benthamiana (SEQ ID NO: 1)

(SEQ ID NO: 1)
AARRPPPPPPTSEEKKDPNMSGVMAKVLASKRRKEAMKESIAKLREKGK
PVKEPSQ

A third blast search performed as in example 3 but using the extended common motif (SEQ ID NO: 13) as query yielded 317 hits of which only a few were longer than 200 amino acids. Table 3 below shows the 20 first hits:

TABLE 3
Results of BLAST search 3

| | | Query | Per. | Acc. | |
|---|---|---|---|---|---|
| Accession | Description | Cover | ident | Len | nr |
| TMW88364.1 | uncharacterized protein | 100% | 100.00% | 130 | 1 |
| | LOC107007224 [Solanum | | | | |
| | pennelli] | | | | |
| XP_006343159.1 | hypothetical protein | 100% | 100.00% | 123 | 2 |
| | EJD97_018654 [Solanum | | | | |
| | chilense] | | | | |
| XP_004252001.1 | PREDICTED: uncharacterized | 100% | 100.00% | 128 | 3 |
| | protein LOC102595503 | | | | |
| | [Solanum tuberosum] | | | | |
| XP_016571877.1 | uncharacterized protein | 100% | 100.00% | 130 | 4 |
| | LOC101243677 [Solanum | | | | |
| | lycopersicum] | | | | |
| XP_016571878.1 | PREDICTED: uncharacterized | 100% | 97.37% | 135 | 5 |
| | protein LOC107870011 isoform | | | | |
| | X1 [Capsicum annuum] | | | | |
| KAH0773866.1 | PREDICTED: uncharacterized | 100% | 97.37% | 130 | 6 |
| | protein LOC107870011 isoform | | | | |
| | X2 [Capsicum annuum] | | | | |
| KAH0706434.1 | hypothetical protein | 100% | 100.00% | 120 | 7 |
| | KY290_011003 [Solanum | | | | |
| | tuberosum] | | | | |
| KAF3613377.1 | hypothetical protein | 100% | 100.00% | 120 | 8 |
| | KY289_011510 [Solanum | | | | |
| | tuberosum] | | | | |
| PHT54591.1 | putative NipSnap protein | 100% | 97.37% | 126 | 9 |
| | K02D10.1-like [Capsicum | | | | |
| | annuum] | | | | |
| KAF3613363.1 | hypothetical protein | 100% | 97.37% | 126 | 10 |
| | CQW23_09053 [Capsicum | | | | |
| | baccatum] | | | | |
| KAG5570777.1 | putative thioredoxin-like 1-1, | 100% | 97.37% | 126 | 11 |
| | chloroplastic-like [Capsicum | | | | |
| | annuum] | | | | |
| XP_019259775.1 | hypothetical protein | 100% | 97.37% | 130 | 12 |
| | H5410_060543 [Solanum | | | | |
| | commersonii] | | | | |
| XP_009587876.1 | PREDICTED: uncharacterized | 100% | 94.74% | 116 | 13 |
| | protein LOC109237842 | | | | |
| | [Nicotiana attenuata] | | | | |
| XP_009779147.1 | uncharacterized protein | 100% | 94.74% | 114 | 14 |
| | LOC104085525 [Nicotiana | | | | |
| | tomentosiformis] | | | | |
| XP_031123519.1 | PREDICTED: uncharacterized | 100% | 92.11% | 116 | 15 |
| | protein LOC104228135 | | | | |
| | [Nicotiana sylvestris] | | | | |
| XP_018852329.1 | uncharacterized protein | 100% | 92.11% | 142 | 16 |
| | LOC116026189 [Ipomoea | | | | |
| | triloba] | | | | |
| XP_019160353.1 | uncharacterized protein | 100% | 94.74% | 131 | 17 |
| | LOC109014350 [Juglans regia] | | | | |
| XP_010069234.2 | PREDICTED: uncharacterized | 100% | 92.11% | 142 | 18 |
| | protein LOC109156914 | | | | |
| | [Ipomoea nil] | | | | |
| CAB3457601.1 | uncharacterized protein | 100% | 94.74% | 123 | 19 |
| | LOC104456191 [Eucalyptus | | | | |
| | grandis] | | | | |
| XP_015061235.1 | unnamed protein product | 100% | 92.11% | 121 | 20 |
| | [Digitaria exilis] | | | | |

Among the 317 total hits, sequence identity varied between 61.52% and 100%, where only 6 hits had sequence identities in the 64-70% range. This indicates that a sequence identity of at least 70% will identify all 72 proteins in the relevant plants.

Further, among the hits, coverage % varied between 76% and 100%, where only 2 hits had less than 80% coverage. This indicates that a coverage % of at least 80% will identify all 72 proteins in the relevant plants.

### EXAMPLE 4

### Inactivation of the 72 Protein in Nicotiana benthamiana Plants Increases Resistance to Bacterial Growth

For bacterial growth assay, we used Pseudomonas syringae (the causative agent of the bacterial speck disease). pv tomato DC3000 ΔhQ. 2-3 fully expanded leaves from each plant was syringe-infiltrated with an OD600=0.0002 of bacterial suspension. 2 days post inoculation, leaf discs from infiltrated leaf were homogenized using three technical replicates. Each disc was sterilised with 15% H₂O₂ for 2 minutes, washed twice with sterile water, and dried for 30 minutes. Tissue-lyser was used to grind leaf tissue using metal beads. Serial dilutions of the leaf extract were plated onto LB agar supplemented with rifampicillin (25 μg ml-1) for selection of PtoDC3000(ΔhQ). 1-2 days after incubation of plates at 28° C., colonies were counted. Twelve samples per group was analysed and CFU/cm2 leaves were analysed. In wild type leaves 40 (std 9.8) CFU/cm² was found, and in leaves with reduced 72 protein 18 CFU/cm² (std 8,4) was found (t-test=0.00006). As seen by the reduction of bacterial growth, the inactivation of the expression of the 72 protein also increased the resistance to bacterial pathogens.

### EXAMPLE 5

### Inactivation of the 72 Protein in Arabidopsis thaliana and Solanum tuberosum Gives Similar Increase of ROS as in Nicotiana benthamiana

Arabidopsis thaliana and Solanum tuberosum plants were investigated with regard to ROS production using the methods applied to Nicotiana benthamiana in Example 2 described above. A similar ROS increase as obtained in Nicotiana benthamiana was obtained.

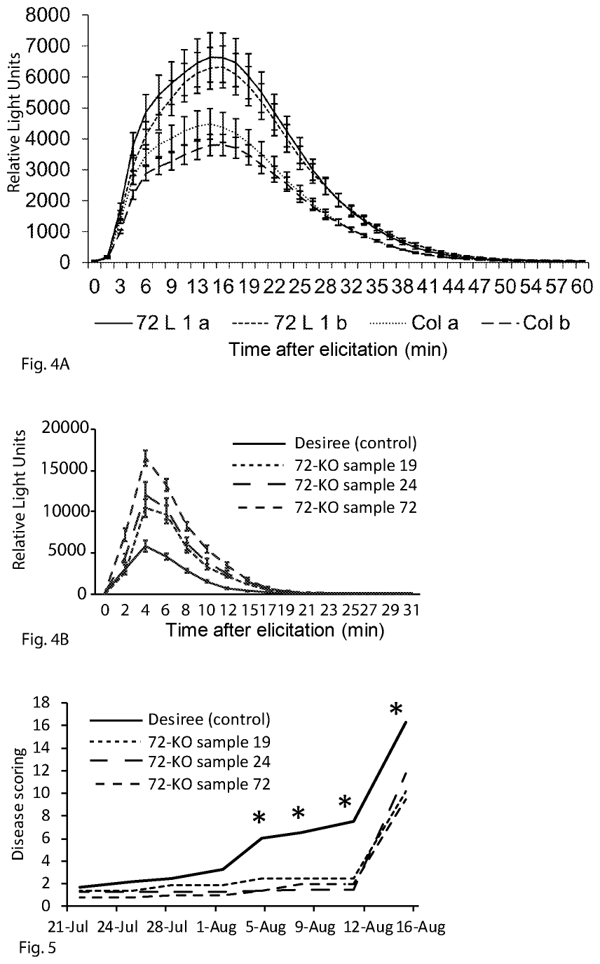

Specifically, FIG. 4A shows the ROS production, as measured in Relative Light Units, of two Arabidopsis thaliana wildtype Colombia (Col A, ColB) plants vs Arabidopsis thaliana Colombia plants with inactivated 72 protein (72 L 1 a, 72 L 1 b) Further, FIG. 4B shows the ROS production, as measured in Relative Light Units, of different Solanum tuberosum 72 protein knock-out lines 19 24 and 72 vs wildtype Solanum tuberosum Desiree plants.

No growth problems were observed with permanent deletion of the gene for the 72 protein using T-DNA insertion.

The 72 protein (immature, i.e. with signal peptide) in Arabidopsis thaliana has the following sequence:

(SEQ ID NO: 15)
MVAHSLVPLSPAAHAARLSSPSPRSLPQAPPVVLAVPPINRRTILVGLG
GALWSWNALAAKEEAMAAARRPPPPPPKEKKDPTVTGVQAKVLASKKRK
EEMKASIAKLREKGKPVVEAKPSSSSSE

### EXAMPLE 6

### Inactivation of the 72 Protein in Nicotiana benthamiana Makes the Plant More Resistant to Dickeya dadantii 3937 Soft Rot Bacteria

As shown in the below table 4, silencing the 72 protein in Nicotiana benthamiana results in an increased resistance to Dickeya dadantii 3937 Soft rot bacteria, as evidenced by a smaller mean lesion size:

TABLE 4
Lesion size

| | | Leaf with knockout |
|---|---|---|
| Parameter | Control leaf | 72 protein |
| Mean lesion size | 24.40741 | 16.68 |
| StdDev | 9.46353 | 9.25887 |
| SEM | 1.821257 | 1.851773 |

P = 0.013 (T-test)

### EXAMPLE 7

### Inactivation of the 72 Protein in Solanum tuberosum Provides Increased Resistance to Late Blight-field Trials

Field trials were carried by planting Solanum tuberosum plants in a field in Borgeby, Sweden during the summer of 2021. 72 Protein knockout plants (lines 19, 24 and 72) and Desiree-WT controls were cultivated in a random block design with four repeats. The field was scored every third to fourth day following the initial identification of P. infestans symptoms in the field, and scoring continued until the end of the season.

The disease scoring showed that the 72 protein knockout plants showed significantly less disease severity as compared to Desiree-WT controls, see FIG. 5, where dashed lines show the results for the respective knockout plants and the solid line shows the results for the Desiree-wt control. Percent yield in late blight untreated plots vs fungicide-treated plots were 52% for Desiree-WT, whereas for the knock-out lines it was 60%, 97% and 88%, for lines 19, 24 and 72, respectively.

Furthermore, no difference in plant height or any other noticeable difference was found between the knockout plants and the control plants. Specifically, the average plant height was: 43±1 cm (Desiree wild type), 41±2 (knock-out line 19), 44±2 cm (knock-out line 24), and 41±2 cm (knock-out line 72).

### EXAMPLE 8

### Inactivation of the 72 Protein Provides Increased Salt Tolerance in Solanum tuberosum Plants

Solanum tuberosum plants in which expression of the 72 protein had been inactivated (knockout) were assayed for ability to grow under salt conditions. The following results were obtained, see table 5:

TABLE 5
Salt tolerance

| Growth condition | Mean root length | P-value t-test vs control |
|---|---|---|
| Desiree-wt (control) at 60 | 4.408857143 | |
| mM NaCl | | |
| 72 Protein knockout line 24 | 10.375 | 2.3647E−5 |
| at 60 mM NaCl | | |
| 72 Protein knockout line 19 | 10.03342857 | 0.00020425 |
| at 60 mM NaCl | | |
| 72 Protein knockout line 72 | 9.758571429 | 0.00074307 |
| at 60 mM NaCl | | |

As seen from the table, the 72 protein knockout plants had longer mean root length in the saline growth conditions than the control.

### EXAMPLE 9

### Inactivation of the 72 Protein in Solanum tuberosum Provides Increased Resistance Towards Alternaria solani

Alternaria solani strain 112 were grown on 20% potato dextrose agar medium incubated in the dark at 25° C. After 7 days, plates were incubated an additional 7 days under UV-c light (model OSRAM HNS15G13 with dominant wavelength 254 nm) for 6 h per day to increase sporulation. The conidia were harvested by flooding the plates with autoclaved tap water containing 0.01% (v/v) Tween 20. The final concentration was adjusted with sterile tap water to 100,000 conidia/ml. 5 weeks old plants (Desire wild type and 72 protein knockout mutant) plants were infected and scored according to as described in [6]. Briefly, 4 to 6 inoculation droplets of 10 μL conidial suspension (100,000 conidia/ml) was placed on the surface of fully development leaf. 2 leaves for one plant and 5 plants per one line. Plants were placed in custom made acrylic glass boxes (422×422×306 mm) with a tray insert (30 mm high) to allow 1 L water to be placed in the bottom without the plants directly touching the water. The infection boxes were closed (keep RH>95%) and placed in Panasonic versatile environmental test chambers (model MLR-352H-PE) equipped with 15 Panasonic FL40SS ENW/37 lights. The incubators were programmed as follows: 06:00-22:00, 25° C., 3 lights on, 0 RH; 22:00-06:00, 22° C., 0 lights on, 0 RH; The experiment was arranged in two test chambers, 4 plants per box and the plants were placed in the boxes in a completely randomized order. Results were recorded by measuring the infection size of each leaf at 5 days post-inoculation (dpi). The difference between the means was tested using a t-test with the significance level of p<0.05 or 0.01.

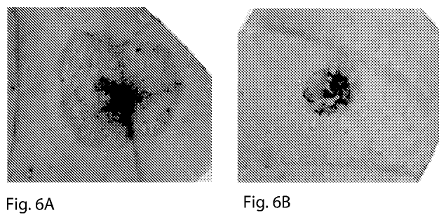

Knocking out the 72 protein conferred disease resistance to Alternaria solani in Solanum tuberosum, see FIG. 6A and FIG. 6B where the lesion caused by Alternaria solani is of much smaller diameter (average lesion diameter of 1-6 mm) and area in FIG. 6B (which shows a leaf from the Solanum tuberosum plant in which the 72 protein was knocked out) than in FIG. 6A (average lesion diameter 11-12 mm) which shows a leaf from the wild type Desiree plant. Average plant height was similar for both knockout plants and control plants at 13 cm whereas fresh biomass weight and dry biomass weight was slightly higher for knockout plants at 80-90 g vs 70 and 16-18 g vs 15 g, respectively.

### REFERENCES

1. Kapila, J.; De Rycke, R.; Van Montagu, M.; Angenon, G. An Agrobacterium-mediated transient gene expression system for intact leaves. Plant Science 1997, 122, 101-108, doi: https://doi.org/10.1016/S0168-9452(96)04541-4.
2. Senthil-Kumar, M.; Mysore, K. S. Tobacco rattle virus-based virus-induced gene silencing in Nicotiana benthamiana. Nature Protocols 2014, 9, 1549-1562, doi: 10.1038/nprot.2014.092.
3. Kourelis, J.; Malik, S.; Mattinson, O.; Krauter, S.; Kahlon, P. S.; Paulus, J. K.; van der Hoorn, R. A. L. Evolution of a guarded decoy protease and its receptor in solanaceous plants. Nature Communications 2020, 11, 4393, doi: 10.1038/s41467-020-18069-5.
4. Sang, Y.; Macho, A. P. Analysis of PAMP-Triggered ROS Burst in Plant Immunity. In Plant Pattern Recognition Receptors: Methods and Protocols, Shan, L., He, P., Eds. Springer New York: New York, NY, 2017; 10.1007/978-1-4939-6859-6_11pp. 143-153.
5. McLellan, H.; Boevink, P. C.; Armstrong, M. R.; Pritchard, L.; Gomez, S.; Morales, J.; Whisson, S. C.; Beynon, J. L.; Birch, P. R. J. An RxLR Effector from Phytophthora infestans Prevents Re-localisation of Two Plant NAC Transcription Factors from the Endoplasmic Reticulum to the Nucleus. PLOS Pathogens 2013, 9, e1003670, doi: 10.1371/journal.ppat.1003670.
6. Brouwer, S. M.; Odilbekov, F.; Burra, D. D.; Lenman, M.; Hedley, P. E.; Grenville-Briggs, L.; Alexandersson, E.; Liljeroth, E.; and Andreasson, E. Intact salicylic acid signalling is required for potato defence against the necrotrophic fungus Alternaria solani. Plant Molecular Biology 2020, 104, 1-19.

### FEASIBLE MODIFICATIONS

The technology proposed herein is not limited only to the embodiments described above and shown in the drawings, which primarily have an illustrative and exemplifying purpose. This patent application is intended to cover all adjustments and variants of the preferred embodiments described herein, thus the present invention is defined by the wording of the appended claims and the equivalents thereof. For instance, it shall be pointed out that structural aspects of embodiments of the methods of the technology proposed herein shall be considered to be applicable to embodiments of the plant cells and plants of the technology proposed herein, and vice versa. It shall also be pointed out that even though it is not explicitly stated that features from a specific embodiment may be combined with features from another embodiment, the combination shall be considered obvious, if the combination is possible. Throughout this specification and the claims which follows, unless the context requires otherwise, the word “comprise”, and variations such as “comprises” or “comprising”, will be understood to imply the inclusion of a stated integer or steps or group of integers or steps but not the exclusion of any other integer or step or group of integers or steps.

1. A method of obtaining a plant cell having increased pathogen resistance and/or abiotic stress tolerance, the method comprising the steps of: a) providing a plant cell, and b) modifying the genome of said plant cell so as to obtain a decreased or inactivated expression of a protein having an amino acid sequence in which at least one part of the amino acid sequence has at least 78%, such as at least 85%, more preferably at least 90% sequence identity to SEQ ID NO: 5, and wherein the protein comprises less than 200 amino acids.

2. The method according to claim 1, wherein the protein has an amino acid sequence in which at least one part of the amino acid sequence has at least 70%, preferably at least 80%, more preferably at least 90% sequence identity to SEQ ID NO: 13, and/or wherein the protein has an amino acid sequence having at least 80%, more preferably at least 90% coverage to SEQ ID NO: 13.

3. The method according to claim 1, wherein the protein has an amino acid sequence with at least 30%, such as at least 40%, more preferably at least 47% sequence identity to SEQ ID NO: 1 or to SEQ ID NO: 3.

4. The method according to claim 1, wherein step (b) of modifying the genome of said plant cell comprises modifying the genome so as to fully or partially decrease or inactivate the expression of a gene sequence in said genome, said gene sequence coding for said protein and preferably having at least 90%, such as at least 95%, more preferably at least 99% sequence identity to SEQ ID NO: 6 or SEQ ID NO: 7.

5. The method according to claim 4, wherein step (b) of modifying the genome of said plant cell comprises mutating or excising at least a part of said gene sequence, preferably using CRISPR/Cas9.

6. The method according to claim 1, wherein the plant cell is selected from the group consisting of a monocot and dicot cell, or wherein the plant cell is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, sugar beet, grape, Arabidopsis and safflower cell, and wherein preferably the plant cell is from the family Solanaceae, preferably from the genus Solanum, more preferably from the species Solanum tuberosum.

7. The method according to claim 1, wherein the increased pathogen resistance comprises increased resistance to at least one pathogen selected from the group consisting of Phytophthora infestans, Dickeya dadantii, and Alternaria solani, and wherein the abiotic stress tolerance comprises tolerance towards at least one abiotic stress selected from the group consisting of salt and drought.

8. A method of obtaining a plant having increased pathogen resistance and/or abiotic stress tolerance, the method comprising the steps a) performing the method according to claim 1 to obtain a plant cell having increased pathogen resistance and/or abiotic stress tolerance, and b) cultivating said plant cell to obtain said plant.

9. A plant cell having increased pathogen resistance and/or abiotic stress tolerance, wherein the genome of the plant cell has been modified to obtain a decreased or inactivated expression of a protein having an amino acid sequence in which at least one part of the amino acid sequence has at least 78%, such as at least 85%, more preferably at least 90% sequence identity to SEQ ID NO: 5, and wherein the protein comprises less than 200 amino acids.

10. The plant cell according to claim 9, wherein the protein has an amino acid sequence in which at least one part of the amino acid sequence has at least 70%, preferably at least 80%, more preferably at least 90% sequence identity to SEQ ID NO: 13, and/or wherein the protein has an amino acid sequence having at least 80%, more preferably at least 90% coverage to SEQ ID NO: 13.

11. The plant cell according to claim 9, wherein the expression of the protein is decreased or inactivated by fully or partially decreasing or inactivating the expression of a gene sequence in said genome, said gene sequence coding for said protein and preferably having at least 90%, such as at least 95%, more preferably at least 99% sequence identity to SEQ ID NO: 6 or SEQ ID NO: 7.

12. The plant cell according to claim 10, wherein the increased pathogen resistance comprises increased resistance to at least one pathogen selected from the group consisting of Phytophthora infestans, Dickeya dadantii, and Alternaria solani, and wherein the abiotic stress tolerance comprises tolerance towards at least one abiotic stress selected from the group consisting of salt and drought.

13. A plant comprising or consisting of one or more plant cells according to claim 1.

14. The plant according to claim 13, wherein the plant is selected from the group consisting of a monocot and dicot plant, or wherein the plant is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, sugar beet, grape, Arabidopsis and safflower cell, and wherein preferably the plant is from the family Solanaceae, preferably from the genus Solanum, more preferably from the species Solanum tuberosum.
